(19) United States
(12) Patent Application Publication
(10) Pub. No.: US 2013/0281376 A1
(43) Pub. Date: Oct. 24, 2013

(54) SPARC MICROENVIRONMENT SIGNATURE, PLASMA SPARC, AND LDH AS PROGNOSTIC BIOMARKERS IN THE TREATMENT OF CANCER

(71) Applicants: Vuong Trieu, Agoura Hills, CA (US); Larn Hwang, Arcadia, CA (US); Kouros Motamed, Irvine, CA (US); Xiping Liu, Temple City, CA (US); Neil Desai, Los Angeles, CA (US)

(72) Inventors: Vuong Trieu, Agoura Hills, CA (US); Larn Hwang, Arcadia, CA (US); Kouros Motamed, Irvine, CA (US); Xiping Liu, Temple City, CA (US); Neil Desai, Los Angeles, CA (US)

(73) Assignee: ABRAXIS BIOSCIENCE, LLC, Los Angeles, CA (US)

(21) Appl. No.: 13/878,067

(22) PCT Filed: Oct. 7, 2011
(86) PCT No.: PCT/US2011/055367

§ 371 Date: Jul. 8, 2013

Related U.S. Application Data
(60) Provisional application No. 61/391,499, filed on Oct. 8, 2010.

(51) Int. Cl. G01N 33/68

(52) U.S. Cl. CPC G01N 33/6893
USPC 514/15.2; 435/7.9; 514/449

(57) ABSTRACT

The invention provides multiparametric anti-SPARC antibody-based techniques for treating cancers as well as determining prognosis and predicting the response to therapy, including chemotherapy, radiotherapy, surgical therapy and combination therapies.

### CROSS-REFERENCE TO RELATED APPLICATIONS

This patent application claims the benefit of U.S. Provisional Patent Application No. 61/391,499, filed Oct. 8, 2011, which is incorporated by reference.

### BACKGROUND OF THE INVENTION

Secreted protein acidic and rich in cysteine (also known as osteonectin, BM40, or SPARC) (hereinafter “SPARC”), is a matrix-associated protein that elicits changes in cell shape, inhibits cell-cycle progression, and influences the synthesis of extracellular matrix (Bradshaw et al., Proc. Nat. Acad. Sci. USA 100: 6045-6050 (2003)). The murine SPARC gene was cloned in 1986 (Mason et al., EMBO J. 5: 1465-1472 (1986)) and a full-length human SPARC cDNA was cloned and sequenced in 1987 (Swaroop et al., Genomics 2: 37-47 (1988)). SPARC expression is developmentally regulated, and is predominantly expressed in tissues undergoing remodeling during normal development or in response to injury. For example, high levels of SPARC protein are expressed in developing bones and teeth (see, e.g., Lane et al., FASEB J., 8, 163 173 (1994); Yan & Sage, J. Histochem. Cytochem. 47:1495-1505 (1999)).

SPARC is upregulated in several aggressive cancers, but is absent in the corresponding normal tissues (Porter et al., J. Histochem. Cytochem., 43, 791 (1995)). SPARC expression is induced among a variety of tumors (e.g., bladder, liver, ovary, kidney, gut, and breast). In bladder cancer, for example, SPARC expression has been associated with advanced carcinoma. Invasive bladder tumors of stage T2 or greater have been shown to express higher levels of SPARC relative to bladder tumors of stage T1 (or less superficial tumors), and a poorer prognosis (see, e.g., Yamanaka et al., J. Urology, 166, 2495 2499 (2001)). In meningiomas, SPARC expression has been associated only with invasive tumors (see, e.g., Rempel et al., Clincal Cancer Res., 5, 237 241 (1999)). SPARC expression also has been detected in 74.5% of in situ invasive breast carcinoma lesions (see, e.g., Bellahcene, et al., Am. J. Pathol., 146, 95 100 (1995)), and 54.2% of infiltrating ductal carcinoma of the breast (see, e.g., Kim et al., J. Korean Med. Sci., 13, 652 657 (1998)). SPARC expression also has been associated with frequent microcalcification in breast cancer (see, e.g., Bellahcene et al., supra), suggesting that SPARC expression may be responsible for the affinity of breast metastases for the bone.

Surprisingly, SPARC has also been shown to have anti-tumor activity in some systems. SPARC is a potent cell cycle inhibitor that arrests cells in mid-G1 (Yan & Sage, J. Histochem. Cytochem. 47:1495-1505 (1999)) and the inducible expression of SPARC has been shown to inhibit breast cancer cell proliferation in an in vitro model system (Dhanesuan et al., Breast Cancer Res. Treat. 75:73-85 (2002)). Similarly, exogenous SPARC can reduce the proliferation of both HOSE (human ovarian surface epithelial) and ovarian cancer cells in a concentration-dependent manner. In addition, SPARC induces apoptosis in ovarian cancer cells. Further, SPARC receptors on cells such as ovarian epithelial cells have been reported. It has been proposed that the binding of SPARC to its receptor is likely to trigger tissue-specific signaling pathways that mediate SPARC's tumor suppressing functions (Yiu et al., Am. J. Pathol. 159:609-622 (2001)). Purified SPARC has also been reported to inhibit angio-gnesis and impair neuroblastoma tumor growth in an in vivo xenograft model system (Chlenski et al., Cancer Res. 62:7357-7363 (2002)).

These seemlingly conflicting results may be due to SPARC's many forms, which result from differential splicing and post translational modifications of immature SPARC. As a result, e.g., fibroblast SPARC is a different molecule than platelet SPARC. In addition, SPARC is differentially glycosylated. (See Kaufman et al., Glycobiology 14(7): 609-619 (2004)). SPARC is readily degraded by a variety of proteases and appears to undergo turnover in extracellular environments. The turnover of SPARC by extracellular proteases results in the exposure of novel SPARC epitopes (Lane & Sage, FASEB J. 8 (2):163-173 (1994)). These factors result in a wide range of immunohistologic staining patterns. Each antibody can produce markedly different staining patterns.

Cancer is now primarily treated with one or a combination of three types of therapies: surgery, radiation, and chemotherapy. Surgery generally is only effective for treating the earlier stages of cancer. For more than 50% of individuals with cancer, by the time they are diagnosed they are no longer candidates for effective surgical treatment. Radiation therapy is only effective for individuals who present with clinically localized disease at early and middle stages of cancer, and is not effective for the late stages of cancer with metastasis.

Chemotherapy involves the disruption of cell replication or cell metabolism. Chemotherapy can be effective, but there are severe side effects, e.g., vomiting, low white blood cells (WBC), hair loss, weight loss and other toxic effects. Because of the extremely toxic side effects, many individuals with cancer cannot successfully finish a complete chemotherapy regime. Chemotherapy-induced side effects significantly impact the quality of life of the individual and may dramatically influence the individual's compliance with treatment. Additionally, adverse side effects associated with chemotherapeutic agents are generally the major dose-limiting toxicity (DLT) in the administration of these drugs. For example, mucositis is a major dose limiting toxicity for several anticancer agents, including the antimetabolite cytotoxic agents 5-FU, methotrexate, and antitumor antibiotics, such as doxorubicin. When severe, many of these chemotherapy-induced side effects may lead to hospitalization, or require treatment with analgesics for pain. Additionally, poor tolerance to chemotherapy can lead to death in some individuals with cancer. The extreme side effects of anticancer drugs are caused by poor target specificity. The drugs circulate through most normal organs as well as the intended target, tumors. The poor target specificity that causes side effects also decreases the efficacy of chemotherapy because only a fraction of the drugs are correctly targeted. The efficacy of chemotherapy is further decreased by poor retention of the anti-cancer drugs within the target tumors.

Due to the severity and breadth of cancer, there is a great need for effective treatments of such diseases or disorders that overcome the shortcomings of surgery, chemotherapy, and radiation treatment. In particular, in view of the serious side effects associated with chemotherapy, there is a need to identify which tumors will or will not respond to chemotherapeutic regimens.

The invention described herein provides novel methods of treating cancer based on the exploitation of the heterogeneous immunohistology observed with different SPARC antibodies.

### BRIEF SUMMARY OF THE INVENTION

The invention provides prognostic and treatment methods for cancerous tumors relating to biomarkers including circulating SPARC levels, SPARC microenvironment signature, and serum lactate dehydrogenase (LDH).

In a first aspect, the invention provides a method of treating a tumor in a mammal with a chemotherapeutic regimen comprising: (a) determining a SPARC microenvironment signature (SMS) of the mammal, wherein the SMS is then compared to a predefined SMS; (b) quantifying plasma SPARC in the mammal; and (c) quantifying plasma lactate dehydrogenase (LDH) in the mammal; and (d) administering a therapeutically effective amount of the chemotherapeutic regimen if two or more of the following conditions is met: SMS satisfies the criteria of a predetermined SMS, circulating SPARC is elevated as compared to a negative control, and plasma LDH is elevated as compared to a negative control.

In another aspect, the invention provides a method of predicting a response to a chemotherapeutic regimen in a mammal comprising: (a) determining a SPARC microenvironment signature (SMS) of the mammal, wherein the SMS is then compared to a predefined SMS; (b) quantifying plasma SPARC in the mammal; and (c) quantifying plasma lactate dehydrogenase (LDH) in the mammal, wherein a positive response to the chemotherapeutic regimen is predicted if two or more of the following conditions are met: SMS satisfies the criteria of a low-risk SMS, circulating SPARC is not elevated as compared to a negative control, and plasma LDH is not elevated as compared to a negative control; and a negative response to the chemotherapeutic regimen is predicted if two or more of the following conditions are met: SMS satisfies the criteria of a high-risk SMS, circulating SPARC is elevated as compared to a negative control, and plasma LDH is elevated as compared to a negative control.

In a further aspect, the invention provides a method of determining a prognosis of a tumor in a mammal comprising: (a) determining a SPARC microenvironment signature (SMS) of the mammal, wherein the SMS is then compared to a predefined SMS; (b) quantifying plasma SPARC in the mammal; and (c) quantifying plasma lactate dehydrogenase (LDH) in the mammal, wherein a positive prognosis is determined if two or more of the following conditions are met: SMS satisfies the criteria of a low-risk SMS, circulating SPARC is not elevated as compared to a negative control, and plasma LDH is not elevated as compared to a negative control; and a negative prognosis is determined if two or more of the following conditions are met: SMS satisfies the criteria of a high-risk SMS, circulating SPARC is elevated as compared to a negative control, and plasma LDH is elevated as compared to a negative control.

The invention further provides exemplary parameters for the SPARC biomarkers. For example, plasma SPARC can be deemed elevated when it exceeds about 366 ng/mL, about 431 ng/mL, or about 495 ng/mL. Plasma LDH can be deemed elevated when it exceeds about 212 IU/mL, about 250 IU/mL, or about 287 IU/mL. For example, when the tumor is breast cancer, the predefined SMS comprises immunostaining with a composite profile with at least 82% of the stroma staining positive with a first antibody and at least a Fibroblast Score of 87, Fibroblast Intensity of 68, Tumor Intensity of 49, Inflammatory Cells Intensity of 42, Inflammatory Cells Score of 67, Blood Vessel % of 68, Tumor Score of 76, Blood Vessel Intensity of 46, Fibroblast % of 51, Blood Vessel Intensity of 75, Inflammatory Cells % of 59, and Stroma Score of 62 staining with a second antibody, wherein the therapy is a regimen comprising nab-paclitaxel and the tumor is pancreatic cancer.

Tumors in the methods of the present invention can be, for example, oral cavity tumors, pharyngeal tumors, digestive system tumors, respiratory system tumors, bone tumors, cartilaginous tumors, bone metastases, sarcomas, skin tumors, melanoma, breast tumors, genital system tumors, urinary tract tumors, orbital tumors, brain and central nervous system tumors, gliomas, endocrine system tumors, thyroid tumors, esophageal tumors, gastric tumors, small intestinal tumors, colonic tumors, rectal tumors, anal tumors, liver tumors, gall bladder tumors, pancreatic tumors, laryngeal tumors, tumors of the lung, bronchial tumors, non-small cell lung carcinoma, small cell lung carcinoma, uterine cervical tumors, uterine corpus tumors, ovarian tumors, vulvar tumors, vaginal tumors, prostate tumors, prostatic carcinoma, testicular tumors, tumors of the penis, urinary bladder tumors, tumors of the kidney, tumors of the renal pelvis, tumors of the ureter, head and neck tumors, parathyroid cancer, Hodgkin's disease, Non-Hodgkin's lymphoma, multiple myeloma, leukemia, acute lymphocytic leukemia, chronic lymphocytic leukemia, acute myeloid leukemia, and chronic myeloid leukemia.

The invention further provides a methods of treatment, prediction of treatment response and outcome based upon a comparison of a primary tumor SMS to the SMS of a metastatic tumor from that primary, with or without incorporating the plasma LDH and/or plasma SPARC levels.

In particular, the invention provides methods for predicting the response of the tumor to a chemotherapeutic regimen, such as a nanoparticulate albumin bound paclitaxel (nab-paclitaxel) and gemcitabine.

Any one of the methods provided by the invention include methods wherein the mammal is a human patient.

### BRIEF DESCRIPTION OF THE SEVERAL VIEWS OF THE DRAWING(S)

FIG. 1 depicts different patterns of SPARC immunostaining generated by two different anti-SPARC antibodies, monoclonal (A), polyclonal (B).

FIG. 2 depicts survival curves for breast cancer patients treated with a nab-paclitaxel based regimen and either expressing the D staining pattern or not.

FIG. 3 depicts a heat diagram from a K-means clustering of progression free survival (PFS) data in breast cancer patients.

FIG. 4A-C depict survival curves reflecting the effect of TN (A), ER (B), and PR (C) status on progression free survival (PFS) in breast cancer.

FIG. 5 depicts survival curves reflecting the effect of the SMS (SPARC microenvironment signature) and TN status on PFS in breast cancer.

FIG. 6 depicts survival curves reflecting the effect of the SMS and ER status on PFS in breast cancer.

FIG. 7 depicts a survival curve reflecting the effect of the SMS and PR status on PFS in breast cancer.

FIG. 8 depicts a heat diagram from a clustering of response data in breast cancer patients using five survival categories.

FIG. 9 depicts a heat diagram from a clustering of response data in breast cancer patients using two survival categories.

FIG. 10 depicts a heat diagram from a K-means clustering of PFS data in pancreatic cancer dividing the patients into good prognosis and bad prognosis SMS groups.

FIG. 11 depicts survival curves for PFS in (A) and overall survival (OS) (B) in pancreatic cancer based on SMS.

FIG. 12 depicts survival curves for PFS in (A) and overall survival (OS) (B) pancreatic cancer based on CA19 status.

FIG. 13 depicts a survival curve reflecting the effect of the SMS and CA 19 status on PFS in pancreatic cancer.

FIG. 14 depicts a survival curve reflecting the effect of the SMS and CA 19 status on OS in pancreatic cancer.

FIG. 15 depicts a heat diagram from a K-means clustering of PFS data in melanoma patients dividing patients into good and bad PFS groups.

FIG. 16 depicts survival curves for PFS in (A) and overall survival (OS) (B) melanoma based on SMS.

FIG. 17A is a plot depicting overall survival of Cohort 1, patients having received prior chemotherapy (PC), for High SPARC and Low SPARC.

FIG. 17B is a plot depicting overall survival of Cohort 2, patients having received no prior chemotherapy (NPC), for High SPARC and Low SPARC.

FIG. 18 is a dot plot depicting SPARC levels in Cohort 1 (prior chemotherapy) and Cohort 2 (no prior chemotherapy), before and after treatment, as compared to normal controls.

FIG. 19 is a bar chart depicting percent change in plasma SPARC following treatment.

FIG. 20A is a plot depicting progression free survival (PFS) for patients in the High Risk cluster (Cluster 1) and Low Risk cluster (Cluster 2).

FIG. 20B is a plot depicting overall survival (OS) for patients in the High Risk cluster (Cluster 1) and Low Risk cluster (Cluster 2).

FIG. 21 is a dot plot depicting SPARC levels in Cohort 1 (prior chemotherapy) and Cohort 2 (no prior chemotherapy), for High Risk (HR) and Low Risk (LR) clusters.

FIG. 22A is a plot depicting progression free survival (PFS) for patients in Risk Levels 0, 1, of 2 as compared to all patients in the High Risk (HR) cluster.

FIG. 22B is a plot depicting overall survival (OS) for patients in Risk Levels 0, 1, of 2 as compared to all patients in the High Risk (HR) cluster.

FIG. 23A is a plot depicting overall survival (OS) for patients having elevated plasma LDH as compared to patients having normal plasma LDH.

FIG. 23B is a plot depicting progression free survival (PFS) for patients having elevated plasma LDH as compared to patients having normal plasma LDH.

FIG. 24A is a plot depicting overall survival (OS) for patients in having 0, 1, or 2 risks.

FIG. 24B is a plot depicting progression free survival (PFS) for patients in Risk Levels 0, 1, of 2 as compared to all patients in the High Risk (HR) cluster.

FIG. 25A is a plot depicting overall survival (OS) for patients in Risk Levels 0, 1, of 2 as compared to all patients in the High Risk (HR) cluster.

FIG. 25B is a plot depicting progression free survival (PFS) for patients in Risk Levels 0, 1, of 2 as compared to all patients in the High Risk (HR) cluster.

FIG. 26 graphically depicts the treatment regimen used in a clinical study demonstrating the effectiveness of a novel combination of nab-paclitaxel, carboplatin and bevacizumab in patients with triple-negative metastatic breast cancer (TNMBC)

FIG. 27 depicts a heat diagram in which the results of immunostaining primary and metastatic tumor components with SPARC antibodies have been clustered according to patient response data with the result that the SPARC SMS of metastatic tumors can discriminate between high and low risk groups.

FIG. 28 graphically demonstrates that the low risk group selectable by SPARC SMS clustering correlates with increased progression free survival relative to the high risk group.

FIG. 29 depicts a heat diagram which shows 8 paired biopsies, from metastatic and primary tumors, obtained from 8 patients in which the SPARC SMS is similar for both metastatic and primary tumors.

FIG. 30 depicts a heat diagram which shows 9 paired biopsies, from metastatic and primary tumors, obtained from 8 patients in which the SPARC SMS for metastatic and primary tumors is dissimilar.

FIG. 31 depicts a heat diagram which compares the individual SPARC SMS components between metastatic and primary tumors in the “similar” group, in which no significant differences among the tumor components are visible.

FIG. 32 depicts a heat diagram that compares the individual SPARC SMS components between metastatic and primary tumors in the “dissimilar” group, in which differences can be seen in the staining of stroma, blood vessels and fibroblasts.

### DETAILED DESCRIPTION OF THE INVENTION

As used herein, the term “cancer” refers to a proliferative disorder caused or characterized by the proliferation of cells which have lost susceptibility to normal growth control. Cancers of the same tissue type usually originate in the same tissue, and may be divided into different subtypes based on their biological characteristics. Four general categories of cancers are carcinoma (epithelial tissue derived), sarcoma (connective tissue or mesodermal derived), leukemia (blood-forming tissue derived) and lymphoma (lymph tissue derived). Over 200 different types of cancers are known, and every organ and tissue of the body may be affected. Specific examples of cancers that do not limit the definition of cancer may include melanoma, leukemia, astrocytoma, glioblastoma, retinoblastoma, lymphoma, glioma, Hodgkins' lymphoma and chronic lymphocyte leukemia. Examples of organs and tissues that may be affected by various cancers include pancreas, breast, thyroid, ovary, uterus, testis, prostate, thyroid, pituitary gland, adrenal gland, kidney, stomach, esophagus or rectum, head and neck, bone, nervous system, skin, blood, nasopharyngeal tissue, lung, urinary tract, cervix, vagina, exocrine glands and endocrine glands. Alternatively, a cancer may be multicentric or of unknown primary site (CUPS).

As used herein, the term “tumor” refers to any neoplastic growth, proliferation or cell mass whether benign or malignant (cancerous), whether a primary site lesion or metastases.

As used herein, a ‘cancerous cell’ refers to a cell that has undergone a transformation event and whose growth is no longer regulated to the same extent as before said transformation event.

As used herein, a “medicament” is a composition capable of producing an effect that may be administered to a patient or test subject. The effect may be chemical, biological or physical, and the patient or test subject may be human, or a non-human animal, such as a rodent or transgenic mouse. The composition may include small organic or inorganic molecules with distinct molecular composition made synthetically, found in nature, or of partial synthetic origin. Included in this group are nucleotides, nucleic acids, amino acids, peptides, polypeptides, proteins, or complexes comprising at least one of these entities, The medicament may be comprised of the effective composition alone or in combination with a pharmaceutically acceptable excipient.

As used herein, a “pharmaceutically acceptable excipient” includes any and all solvents, dispersion media, coatings, antibacterial, antimicrobial or antifungal agents, isotonic and absorption delaying agents, and the like that are physiologically compatible. The excipient may be suitable for intravenous, intraperitoneal, intramuscular, intrathecal or oral administration. The excipient may include sterile aqueous solutions or dispersions for extemporaneous preparation of sterile injectable solutions or dispersion. Use of such media for preparation of medicaments is known in the art.

As used herein, a “effective amount” or a “pharmacologically effective amount” or a “therapeutically effective amount” of a medicament, drug or therapy refers to an amount which upon administration it rearches concentrations in the therapeutic level of the medicament, drug or therapy delivered over the term that it is used. This may be dependent on mode of delivery, time period of the dosage, age, weight, general health, sex and diet of the subject receiving the medicament. The determination of what dose is a “pharmacologically effective amount” requires routine optimization which is within the capabilities of one of ordinary skill in the art. A cancer or cancerous cell may be described as “sensitive to” or “resistant to” a given therapeutic regimen or chemotherapeutic agent based on the ability of the regimen to kill cancer cells or decrease tumor size, reduce overall cancer growth (i.e. through reduction of angio elements), and/or inhibit metastasis. Cancer cells that are resistant to a therapeutic regimen may not respond to the regimen and may continue to proliferate. Cancer cells that are sensitive to a therapeutic regimen may respond to the regimen resulting in cell death, a reduction in tumor size, reduced overall growth (tumor burden) or inhibition of metastasis.

The terms “treating,” “treatment,” “therapy,” and “therapeutic treatment” as used herein refer to curative therapy, prophylactic therapy, or preventative therapy. An example of “preventative therapy” is the prevention or lessening the chance of a targeted disease (e.g., cancer or other proliferative disease) or related condition thereto. Those in need of treatment include those already with the disease or condition as well as those prone to have the disease or condition to be prevented. The terms “treating,” “treatment,” “therapy,” and “therapeutic treatment” as used herein also describe the management and care of a mammal for the purpose of combating a disease, or related condition, and includes the administration of a composition to alleviate the symptoms, side effects, or other complications of the disease, condition. Therapeutic treatment for cancer includes, but is not limited to, surgery, chemotherapy, radiation therapy, gene therapy, and immunotherapy.

As used herein, the term “agent” or “drug” or “therapeutic agent” refers to a chemical compound, a mixture of chemical compounds, a biological macromolecule, or an extract made from biological materials such as bacteria, plants, fungi, or animal (particularly mammalian) cells or tissues that are suspected of having therapeutic properties. The agent or drug can be purified, substantially purified or partially purified. An “agent” according to the present invention, also includes a radiation therapy agent or a “chemotherapuetic agent.”

As used herein, “chemotherapy” refers to the administration of at least one chemotherapy agent which is harmful to destroy cancerous cells. There are a myriad of such chemotherapy agents available to a clinician. Chemotherapy agents may be administered to a subject in a single bolus dose, or may be administered in smaller doses over time. A single chemotherapeutic agent may be used (single-agent therapy) or more than one agent may be used in combination (combination therapy). Chemotherapy may be used alone to treat some types of cancer. Alternatively, chemotherapy may be used in combination with other types of treatment, for example, radiotherapy or alternative therapies (for example immunotherapy) as described herein. Additionally, a chemosensitizer may be administered as a combination therapy with a chemotherapy agent.

As used herein, a “chemotherapeutic agent” or “anticancer drug” refers to a medicament that may be used to treat cancer, and generally has the ability to kill cancerous cells directly. Examples of chemotherapeutic agents include alkylating agents, antimetabolites, natural products, hormones and antagonists, and miscellaneous agents. Examples of alternate names are indicated in brackets. Examples of alkylating agents include nitrogen mustards such as mechlorethamine, cyclophosphamide, ifosfamide, melphalan (L-sarcolysin) and chlorambucil; ethylenimines and methylmelamines such as hexamethylmelamine and thiotepa; alkyl sulfonates such as busulfan; nitrosoureas such as carmustine (BCNU), semustine (methyl-CCNU), lomustine (CCNU) and streptozocin (streptozotocin); DNA synthesis antagonists such as estramustine phosphate; and triazines such as dacarbazine (DTIC, dimethyl-triazenoimidazolecarboxamide) and temozolomide. Examples of antimetabolites include folic acid analogs such as methotrexate (amethopterin); pyrimidine analogs such as fluorouracin (5-fluorouracil, 5-FU, 5FU), floxuridine (fluorodeoxyuridine, FUdR), cytarabine (cytosine arabinoside) and gemcitabine; purine analogs such as mercaptopurine (6-mercaptopurine, 6-MP), thioguanine (6-thioguanine, TG) and pentostatin (2′-deoxycoformycin, deoxycoformycin), cladribine and fludarabine; and topoisomerase inhibitors such as amsacrine. Examples of natural products include vinca alkaloids such as vinblastine (VLB) and vincristine; taxanes such as paclitaxel and docetaxel (Taxotere); epipodophyllotoxins such as etoposide and teniposide; camptothecins such as topotecan and irinotecan; antibiotics such as dactinomycin (actinomycin D), daunorubicin (daunomycin, rubidomycin), doxorubicin, bleomycin, mitomycin (mitomycin C), idarubicin, epirubicin; enzymes such as L-asparaginase; and biological response modifiers such as interferon alpha and interlelukin 2. Examples of hormones and antagonists include luteinising releasing hormone agonists such as buserelin; adrenocorticosteroids such as prednisone and related preparations; progestins such as hydroxyprogesterone caproate, medroxyprogesterone acetate and megestrol acetate; estrogens such as diethylstilbestrol and ethinyl estradiol and related preparations; estrogen antagonists such as tamoxifen and anastrozole; androgens such as testosterone propionate and fluoxymesterone and related preparations; androgen antagonists such as flutamide and bicalutamide; and gonadotropin-releasing hormone analogs such as leuprolide. Examples of miscellaneous agents include thalidomide; platinum coordination complexes such as cisplatin (cis-DDP), oxaliplatin and carboplatin; anthracenediones such as mitoxantrone; substituted ureas such as hydroxyurea; methylhydrazine derivatives such as procarbazine (N-methylhydrazine, MIH); adrenocortical suppressants such as mitotane (o,p′-DDD) and aminoglutethimide; RXR agonists such as bexarotene; and tyrosine kinase inhibitors such as imatinib. Alternate names and trade-names of these and additional examples of chemotherapeutic agents, and their methods of use including dosing and administration regimens, will be known to a person versed in the art. In particular, suitable chemotherapeutic agents for use in accordance with the invention include, without limitation, nanoparticle albumin-bound paclitaxels.

Abraxane™, also known as ABI-007, is a preferred chemotherapeutic agent. Abraxane™ is an albumin-nanoparticle formulation of paclitaxel. The use of an albumin nanoparticle as a vehicle results in the formation of a colloid when reconstituted with saline. Based on clinical studies, it has been shown that the use of Abraxane™ is characterized by reduced hypersensitivity reactions as compared with Taxol.™ Accordingly, premedication is not required for patients receiving Abraxane™.

Another advantage of the albumin-nanoparticle formulation is that by excluding toxic emulsifiers it is possible to administer higher doses of paclitaxel at more frequent intervals than is possible with Taxol™. The potential exists that enhanced efficacy could be seen in solid tumors as a consequence of (i) higher tolerable doses (300 mg/m²), (ii) longer half-life, (iii) prolonged local tumor availability and/or (iv) sustained in vivo release Abraxane™.

The term “correlated” refers to the dependency based on a Pearson's product-moment coefficient. The term statistically significant means a p-value of 0.05 or less.

A positive response is defined as including, but not limited, to pathological response (reduction in tumor size or burden), overall survival, or progression free survival as shown by an improvement of the metric by at least 5%, preferably by at least 10%, more preferably by at least 15%, even more preferably by at least 20%, most preferably by at least 25% or more. Alternatively, the metric shows an improvement by a statistically significant amount in comparison with no or prior or alternative therapy.

A negative response includes, but is not limited to pathological progression, decreased overall or progression free survival.

As used herein, the term “radiotherapeutic regimen” or “radiotherapy” refers to the administration of radiation to kill cancerous cells. Radiation interacts with various molecules within the cell, but the primary target, which results in cell death is the deoxyribonucleic acid (DNA). However, radiotherapy often also results in damage to the cellular and nuclear membranes and other organelles. DNA damage usually involves single and double strand breaks in the sugar-phosphate backbone. Furthermore, there can be cross-linking of DNA and proteins, which can disrupt cell function. Depending on the radiation type, the mechanism of DNA damage may vary as does the relative biologic effectiveness. For example, heavy particles (i.e. protons, neutrons) damage DNA directly and have a greater relative biologic effectiveness. Electromagnetic radiation results in indirect ionization acting through short-lived, hydroxyl free radicals produced primarily by the ionization of cellular water. Clinical applications of radiation consist of external beam radiation (from an outside source) and brachytherapy (using a source of radiation implanted or inserted into the patient). External beam radiation consists of X-rays and/or gamma rays, while brachytherapy employs radioactive nuclei that decay and emit alpha particles, or beta particles along with a gamma ray.

Radiotherapy may further be used in combination chemotherapy, with the chemotherapeutic agent acting as a radiosensitizer. The specific choice of radiotherapy suited to an individual patient may be determined by a skilled person at the point of care, taking into consideration the tissue and stage of the cancer.

As used herein, the term “alternative therapeutic regimen” or “alternative therapy” may include for example, biologic response modifiers (including polypeptide-, carbohydrate-, and lipid-biologic response modifiers), toxins, lectins, antiangiogenic agents, receptor tyrosine kinase inhibitors (for example Iressa™ (gefitinib), Tarceva™ (erlotinib), Erbitux™ (cetuximab), imatinib mesilate (Gleevec™), proteosome inhibitors (for example bortezomib, Velcade™); VEGFR2 inhibitors such as PTK787 (ZK222584), aurora kinase inhibitors (for example ZM447439); mammalian target of rapamycin (mTOR) inhibitors, cyclooxygenase-2 (COX-2) inhibitors, rapamycin inhibitors (for example sirolimus, Rapamune™); farnesyltransferase inhibitors (for example tipifarnib, Zarnestra); matrix metalloproteinase inhibitors (for example BAY 12-9566; sulfated polysaccharide tecogalan); angiogenesis inhibitors (for example Avastin™ (bevacizumab); analogues of fumagillin such as TNP-4; carboxyaminotriazole; BB-94 and BB-2516; thalidomide; interleukin-12; linomide; peptide fragments; and antibodies to vascular growth factors and vascular growth factor receptors); platelet derived growth factor receptor inhibitors, protein kinase C inhibitors, mitogen-activated kinase inhibitors, mitogen-activated protein kinase kinase inhibitors, Rous sarcoma virus transforming oncogene (SRC) inhibitors, histonedeacetylase inhibitors, small hypoxia-inducible factor inhibitors, hedgehog inhibitors, and TGF-β signalling inhibitors. Furthermore, an immunotherapeutic agent would also be considered an alternative therapeutic regimen. Examples include chemokines, chemotaxins, cytokines, interleukins, or tissue factor. Suitable immunotherapeutic agents also include serum or gamma globulin containing preformed antibodies; nonspecific immunostimulating adjuvants; active specific immunotherapy; and adoptive immunotherapy. In addition, alternative therapies may include other biological-based chemical entities such as polynucleotides, including antisense molecules, polypeptides, antibodies, gene therapy vectors and the like. Such alternative therapeutics may be administered alone or in combination, or in combination with other therapeutic regimens described herein. Alternate names and trade-names of these agents used in alternative therapeutic regimens and additional examples of agents used in alternative therapeutic regimens, and their methods of use including dosing and administration regimens, will be known to a physician versed in the art. Furthermore, methods of use of chemotherapeutic agents and other agents used in alternative therapeutic regimens in combination therapies, including dosing and administration regimens, will also be known to a person versed in the art.

In particular, suitable alternative therapeutic regimens include, without limitation, antibodies to molecules on the surface of cancer cells such as antibodies to Her2 (e.g., Trastuzumab), EGF or EGF Receptors, VEGF (e.g., Bevacizumab) or VEGF Receptors, CD20, and the like. The therapeutic agent may further comprise any antibody or antibody fragment which mediates one or more of complement activation, cell mediated cytotoxicity, inducing apoptosis, inducing cell death, and opsinization. For example, such an antibody fragment may be a complete or partial Fc domain.

As used herein, the term “histologic section” refers to a thin section of a tissue sample suitable for mounting on a microscope slide and staining with any suitable protocol. As used herein, “immunostaining a histologic section” refers to the staining of the cells and intracellular matrix of the histologic section resulting from the binding of antibodies to components of the cells are intracellular matrix. As used herein, to “predominantly” or “preferentially” stain a structure, e.g., a cancer cell over a fibroblast, the immunostaining of the preferentially stained structure in the histologic section should be of an intensity graded by a pathologist by any suitable system, including, e.g., 3/3 when observed microscopically by those of ordinary skill, well all other structures stain with only an intensity of 1/3 or show 0/3 (no staining).

As used herein, the term “epitope” refers to the three-dimensional structure bound by an antibody, and in particular the amino acid sequence targeted by the antibody. As used herein, the term “epitope recognized by the MAB941 monoclonal antibody” refers to the amino acid sequence in SPARC bound by the MAB941 monoclonal anybody. (SPARC monoclonal antibody (R&D Systems, Minneapolis, Minn.), catalog #MAB941)

As used herein, “immunodominant epitopes” refers to the three-dimensional structures bound with the greatest collective avidity by the antibodies in polyclonal antisera. In particular, the epitopes responsible for the pattern of staining in immunostaining protocol employing that polyclonal antisera. As used herein, the term “immunodominant SPARC epitopes recognized by the AF941 polyconal antibody refers” to the SPARC peptides and amino acid sequences found with the greatest avidity by the AF941 polyconal antisera. Accordingly, binding to and staining of these SPARC peptides and amino acid sequences results and the majority of immunostaining observed. (SPARC polyclonal antibody (R&D Systems, Minneapolis, Minn.), catalog #AF941)

By “antibodies” it is meant without limitation, monoclonal antibodies, polyclonal antibodies, dimers, multimers, multispecific antibodies (e.g., bispecific antibodies). Antibodies may be murine, human, humanized, chimeric, or derived from other species. An antibody is a protein generated by the immune system that is capable of recognizing and binding to a specific antigen. A target antigen generally has numerous binding sites, also called epitopes, recognized by CDRs on multiple antibodies. Each antibody that specifically binds to a different epitope has a different structure. Thus, one antigen may have more than one corresponding antibody.

An antibody includes a full-length immunoglobulin molecule or an immunologically active portion of a full-length immunoglobulin molecule, i.e., a molecule that contains an antigen binding site that immunospecifically binds an antigen of a target of interest or part thereof. Targets include, cancer cells or other cells that produce autoimmune antibodies associated with an autoimmune disease.

The immunoglobulins disclosed herein can be of any class (e.g., IgG, IgE, IgM, IgD, and IgA) or subclass (e.g., IgG1, IgG2, IgG3, IgG4, IgA1 and IgA2) of immunoglobulin molecule. The immunoglobulins can be derived from any species.

“Antibody fragments” comprise a portion of a full length antibody, which maintain the desired biological activity. “Antibody fragments” are generally the antigen binding or variable region thereof. Examples of antibody fragments include Fab, Fab′, F(ab′)2, and Fv fragments; diabodies; linear antibodies; fragments produced by a Fab expression library, anti-idiotypic (anti-Id) antibodies, CDR (complementary determining region), and epitope-binding fragments of any of the above which immunospecifically bind to cancer cell antigens, viral antigens or microbial antigens, single-chain antibody molecules; and multispecific antibodies formed from antibody fragments.

The monoclonal antibodies referenced herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567). Chimeric antibodies of interest herein include “primatized” antibodies comprising variable domain antigen-binding sequences derived from a non-human primate (e.g., Old World Monkey or Ape) and human constant region sequences.

“Antibody-dependent cell-mediated cytotoxicity” and “ADCC” refer to a cell-mediated reaction in which nonspecific cytotoxic cells that express Fc receptors (FcRs) (e.g., Natural Killer (NK) cells, neutrophils, and macrophages) recognize bound antibody on a target cell and subsequently cause lysis of the target cell. The primary cells for mediating ADCC, NK cells, express Fc.γ.RIII only, whereas monocytes express FcγRI, FcγRII and FcγRIII. To assess ADCC activity of a molecule of interest, an in vitro ADCC assay may be performed (U.S. Pat. No. 5,003,621; U.S. Pat. No. 5,821,337). Useful effector cells for such assays include peripheral blood mononuclear cells (PBMC) and Natural Killer (NK) cells.

An antibody which “induces cell death” is one which causes a viable cell to become nonviable. Cell death in vitro may be determined in the absence of complement and immune effector cells to distinguish cell death induced by antibody-dependent cell-mediated cytotoxicity (ADCC) or complement dependent cytotoxicity (CDC). Thus, the assay for cell death may be performed using heat inactivated serum (i.e., in the absence of complement) and in the absence of immune effector cells. To determine whether the antibody is able to induce cell death, loss of membrane integrity as evaluated by uptake of propidium iodide (PI), trypan blue or 7AAD can be assessed relative to untreated cells. Cell death-inducing antibodies are those which induce PI uptake in the PI uptake assay in BT474 cells.

An antibody which “induces apoptosis” is one which induces programmed cell death as determined by binding of annexin V, fragmentation of DNA, cell shrinkage, dilation of endoplasmic reticulum, cell fragmentation, and/or formation of membrane vesicles (called apoptotic bodies).

As used herein, a “chemosensitizer” or “sensitizer” is a medicament that may enhance the therapeutic effect of a chemotherapeutic agent, radiotherapy treatment or alternative therapeutic regimen, and therefore improve efficacy of such treatment or agent. The sensitivity or resistance of a tumor or cancerous cell to treatment may also be measured in an animal, such as a human or rodent, by, e.g., measuring the tumor size, tumor burden or incidence of metastases over a period of time. For example, about 2, about 3, about 4 or about 6 months for a human and about 2-4, about 3-5, or about 4-6 weeks for a mouse. A composition or a method of treatment may sensitize a tumor or cancerous cell's response to a therapeutic treatment if the increase in treatment sensitivity or the reduction in resistance is about 10% or more, for example, about 30%, about 40%, about 50%, about 60%, about 70%, about 80%, or more, to about 2-fold, about 3-fold, about 4-fold, about 5-fold, about 10-fold, about 15-fold, about 20-fold or more, compared to treatment sensitivity or resistance in the absence of such composition or method. The determination of sensitivity or resistance to a therapeutic treatment is routine in the art and within the skill of a person versed in the art.

The terms ‘“peptide,” “polypeptide,” and “protein” may be used interchangeably, and refer to a compound comprised of at least two amino acid residues covalently linked by peptide bonds or modified peptide bonds, for example peptide isosteres (modified peptide bonds) that may provide additional desired properties to the peptide, such as increased half-life. A peptide may comprise at least two amino acids. The amino acids comprising a peptide or protein described herein may also be modified either by natural processes, such as posttranslational processing, or by chemical modification techniques which are well known in the art. Modifications can occur anywhere in a peptide, including the peptide backbone, the amino acid side-chains and the amino or carboxyl termini. It is understood that the same type of modification may be present in the same or varying degrees at several sites in a given peptide.

The invention provides a method of treating a tumor in a mammal with a chemotherapeutic regimen comprising: (a) determining a SPARC microenvironment signature (SMS) of the mammal, wherein the SMS is then compared to a predefined SMS; (b) quantifying plasma SPARC in the mammal; and (c) quantifying plasma lactate dehydrogenase (LDH) in the mammal; and (d) administering a therapeutically effective amount of the chemotherapeutic regimen if two or more of the following conditions is met: SMS satisfies the criteria of a predetermined SMS, circulating SPARC is elevated as compared to a negative control, and plasma LDH is elevated as compared to a negative control. In a preferred embodiment, all three of the foregoing conditions are met.

The invention also provides a method of predicting a response to a chemotherapeutic regimen in a mammal comprising: (a) determining a SPARC microenvironment signature (SMS) of the mammal, wherein the SMS is then compared to a predefined SMS; (b) quantifying plasma SPARC in the mammal; and (c) quantifying plasma lactate dehydrogenase (LDH) in the mammal, wherein a positive response to the chemotherapeutic regimen is predicted if two or more of the following conditions are met: SMS satisfies the criteria of a low-risk SMS, circulating SPARC is not elevated as compared to a negative control, and plasma LDH is not elevated as compared to a negative control. In a preferred embodiment, a positive response to the chemotherapeutic regimen is predicted if all three of the preceding conditions are met. A negative response to the chemotherapeutic regimen is predicted if two or more of the following conditions are met: SMS satisfies the criteria of a high-risk SMS, circulating SPARC is elevated as compared to a negative control, and plasma LDH is elevated as compared to a negative control. In a preferred embodiment, a negative response to the chemotherapeutic regimen is predicted if all three of the preceding conditions are met.

Additionally, the invention provides a method of determining a prognosis, i.e., likelihood of progression, of a tumor in a mammal comprising: (a) determining a SPARC microenvironment signature (SMS) of the mammal, wherein the SMS is then compared to a predefined SMS; (b) quantifying plasma SPARC in the mammal; and (c) quantifying plasma lactate dehydrogenase (LDH) in the mammal, wherein a positive prognosis is determined if two or more of the following conditions are met: SMS satisfies the criteria of a low-risk SMS, circulating SPARC is not elevated as compared to a negative control, and plasma LDH is not elevated as compared to a negative control. In a preferred embodiment, a positive prognosis is determined if all three of the preceding conditions are met. A negative prognosis is determined if two or more of the following conditions are met: SMS satisfies the criteria of a high-risk SMS, circulating SPARC is elevated as compared to a negative control, and plasma LDH is elevated as compared to a negative control. In a preferred embodiment, a negative prognosis is determined if all three of the preceding conditions are met.

Methods of determining an SMS are detailed infra, and are also described in PCT/US2010/049545, filed September 2010, which is incorporated herein by reference. Plasma (i.e., circulating) SPARC can be quantified by any suitable method known to one of ordinary skill in the art, such as by ELISA. Plasma LDH levels can be determined by any suitable method known to one of ordinary skill in the art, and can be obtained by standard diagnostic methods.

Plasma LDH can be considered “elevated” according to the methods of the present invention if it exceeds plasma LDH levels typically found in a negative control, i.e., a healthy mammal of the same species. In humans, plasma LDH can be considered “elevated” if it exceeds about 212 IU/mL. Preferably, plasma LDH is considered “elevated” if it exceeds about 250 IU/mL. More preferably, plasma LDH is considered “elevated” if it exceeds about 287 IU/mL. It will be understood by one of ordinary skill in the art that these plasma LDH levels represent a level of about 250 IU/mL, plus or minus about 15%, as is conventional in bioanalytical methods.

Likewise, plasma SPARC can be considered “elevated” according to the methods of the present invention if it exceeds plasma SPARC levels typically found in a negative control, i.e., a healthy mammal of the same species. In humans, plasma SPARC can be considered “elevated” if it exceeds about 366 ng/mL. Preferably, plasma SPARC is considered “elevated” if it exceeds about 431 ng/mL. More preferably, plasma SPARC is considered “elevated” if it exceeds about 495 ng/mL. It will be understood by one of ordinary skill in the art that these plasma SPARC levels represent a level of about 431 IU/mL, plus or minus about 15%, as is conventional in bioanalytical methods.

In the methods of the present invention, a prognosis or method of treatment can be determined when two or more of the biomarkers discussed herein are in agreement. For example, a negative prognosis of a tumor is indicated, and a particular chemotherapeutic treatment would be considered less likely to be successful, when plasma SPARC and LDH are elevated as compared to a negative control. These findings would also exist when the SMS satisfies the criteria of a high risk SMS in combination with either elevated plasma SPARC or elevated plasma LDH. Of course, a high risk SMS in combination with both elevated plasma SPARC and elevated plasma LDH would also indicate a negative prognosis of a tumor (i.e., a likelihood that the tumor would progress) as well as that a particular chemotherapeutic treatment would be considered less likely to be successful.

Conversely, a positive prognosis of a tumor is indicated, and a particular chemotherapeutic treatment would be considered more likely to be successful, when plasma SPARC and LDH are not elevated as compared to a negative control. These findings would also exist when the SMS satisfies the criteria of a low-risk SMS in combination with plasma SPARC or plasma LDH that are normal (i.e., not elevated as compared to a negative control). Similarly, a low-risk SMS in combination with normal plasma SPARC and normal plasma LDH would also indicate a positive prognosis of a tumor as well as a likelihood of success of a particular chemotherapeutic treatment.

Predefined SMS in which a particular chemotherapeutic regimen is thought to be effective can be prepared using the methods described infra. However, one exemplary SMS, associated with breast cancer, comprises immunostaining with a composite profile with at least 82% of the stroma staining positive with a first antibody and at least a Fibroblast Score of 87, Fibroblast Intensity of 68, Tumor Intensity of 49, Inflammatory Cells Intensity of 42, Inflammatory Cells Score of 67, Blood Vessel % of 68, Tumor Score of 76, Blood Vessel Intensity of 46, Fibroblast % of 51, Blood Vessel Intensity of 75, Inflammatory Cells % of 59, and Stroma Score of 62 staining with a second antibody. Such SMS indicates that the tumor is likely to respond to a chemotherapeutic regimen comprising nab-paclitaxel and the tumor is pancreatic cancer. However, other SMS can be prepared using tumor samples taken from subjects prior to treatment and then retroactively associated with effective chemotherapeutic regimens.

Any cancerous tumor can be evaluated and/or treated according to the methods of the present invention. Examples of contemplated tumors include oral cavity tumors, pharyngeal tumors, digestive system tumors, respiratory system tumors, bone tumors, cartilaginous tumors, bone metastases, sarcomas, skin tumors, melanoma, breast tumors, genital system tumors, urinary tract tumors, orbital tumors, brain and central nervous system tumors, gliomas, endocrine system tumors, thyroid tumors, esophageal tumors, gastric tumors, small intestinal tumors, colonic tumors, rectal tumors, anal tumors, liver tumors, gall bladder tumors, pancreatic tumors, laryngeal tumors, tumors of the lung, bronchial tumors, non-small cell lung carcinoma, small cell lung carcinoma, uterine cervical tumors, uterine corpus tumors, ovarian tumors, vulvar tumors, vaginal tumors, prostate tumors, prostatic carcinoma, testicular tumors, tumors of the penis, urinary bladder tumors, tumors of the kidney, tumors of the renal pelvis, tumors of the ureter, head and neck tumors, parathyroid cancer, Hodgkin's disease, Non-Hodgkin's lymphoma, multiple myeloma, leukemia, acute lymphocytic leukemia, chronic lymphocytic leukemia, acute myeloid leukemia, and chronic myeloid leukemia. Preferably the tumor is a pancreatic cancer.

The mammal can be any patient or subject in need of treatment or diagnosis. In particularly preferred embodiments, the mammal is a human. In other embodiments, the mammal can be a mouse, rat, rabbit, cat, dog, pig, sheep, horse, cow, or a non-human primate.

In other embodiments, the inventive methods comprise administering to a mammal a therapeutically effective amount of a pharmaceutical composition comprising paclitaxel. In particularly preferred embodiments, the composition comprises nab-paclitaxel (Abraxane®).

One or more doses of one or more chemotherapeutic agents, such as those described above, can also be administered according to the inventive methods. The type and number of chemotherapeutic agents used in the inventive method will depend on the standard chemotherapeutic regimen for a particular tumor type. In other words, while a particular cancer can be treated routinely with a single chemotherapeutic agent, another can be treated routinely with a combination of chemotherapeutic agents. Methods for coupling or conjugation of suitable therapeutics, chemotherapeutics, radionuclides, etc. to antibodies or fragments thereof are well described in the art. The following examples further illustrate the invention but, of course, should not be construed as in any way limiting its scope.

Methods in accordance with the invention include, e.g., combination therapies wherein the animal is also undergoing one or more cancer therapies selected from the group consisting of surgery, chemotherapy, radiotherapy, thermotherapy, immunotherapy, hormone therapy and laser therapy. The terms “co-administration” and “combination therapy” refer to administering to a subject two or more therapeutically active agents. The agents can be contained in a single pharmaceutical composition and be administered at the same time, or the agents can be contained in separate formulation and administered serially to a subject. So long as the two agents can be detected in the subject at the same time, the two agents are said to be co-administered.

Therapies contemplated in the treatment methods of the present invention include, but are not limited to antibody administration, vaccine administration, administration of cytotoxic agents, natural amino acid polypeptides, nucleic acids, nucleotide analogues, and biologic response modifiers. Two or more combined compounds may be used together or sequentially. Examples of chemotherapeutic agents include alkylating agents, antimetabolites, natural products, hormones and antagonists, and miscellaneous agents. Examples of alkylating agents include nitrogen mustards such as mechlorethamine, cyclophosphamide, ifosfamide, melphalan (L-sarcolysin) and chlorambucil; ethylenimines and methylmelamines such as hexamethylmelamine and thiotepa; alkyl sulfonates such as busulfan; nitrosoureas such as carmustine (BCNU), semustine (methyl-CCNU), lomustine (CCNU) and streptozocin (streptozotocin); DNA synthesis antagonists such as estramustine phosphate; and triazines such as dacarbazine (DTIC, dimethyl-triazenoimidazolecarboxamide) and temozolomide. Examples of antimetabolites include folic acid analogs such as methotrexate (amethopterin); pyrimidine analogs such as fluorouracin (5-fluorouracil, 5-FU, 5FU), floxuridine (fluorodeoxyuridine, FUdR), cytarabine (cytosine arabinoside) and gemcitabine; purine analogs such as mercaptopurine (6-niercaptopurine, 6-MP), thioguanine (6-thioguanine, TG) and pentostatin (2′-deoxycoformycin, deoxycoformycin), cladribine and fludarabine; and topoisomerase inhibitors such as amsacrine. Examples of natural products include vinca alkaloids such as vinblastine (VLB) and vincristine; taxanes such as paclitaxel (Abraxane®) and docetaxel (Taxotere®); epipodophyllotoxins such as etoposide and teniposide; camptothecins such as topotecan and irinotecan; antibiotics such as dactinomycin (actinomycin D), daunorubicin (daunomycin, rubidomycin), doxorubicin, bleomycin, mitomycin (mitomycin C), idarubicin, epirubicin; enzymes such as L-asparaginase; and biological response modifiers such as interferon alpha and interlelukin 2. Examples of hormones and antagonists include luteinising releasing hormone agonists such as buserelin; adrenocorticosteroids such as prednisone and related preparations; progestins such as hydroxyprogesterone caproate, medroxyprogesterone acetate and megestrol acetate; estrogens such as diethylstilbestrol and ethinyl estradiol and related preparations; estrogen antagonists such as tamoxifen and anastrozole; androgens such as testosterone propionate and fluoxymesterone and related preparations; androgen antagonists such as flutamide and bicalutamide; and gonadotropin-releasing hormone analogs such as leuprolide. Examples of miscellaneous agents include thalidomide; platinum coordination complexes such as cisplatin (czs-DDP), oxaliplatin and carboplatin; anthracenediones such as mitoxantrone; substituted ureas such as hydroxyurea; methylhydrazine derivatives such as procarbazine (N-methylhydrazine, MIH); adrenocortical suppressants such as mitotane (o,p′-DDD) and aminoglutethimide; RXR agonists such as bexarotene; and tyrosine kinase inhibitors such as imatinib.

In preparing SMS according to the methods of the present invention, a tissue array can first be made and stained by any suitable method known to those of ordinary skill in the art. For example, tissue cores from formalin-fixed, paraffin-embedded tumor blocks (2 cores from the most representative areas per block) can be arrayed (Beecher Instruments, Silver Spring, Md.) to create a tissue microarray of cores measuring 2.0 mm each and were placed on positively charged slides. Slides with specimens are then placed in a 60° C. oven for 1 hour, cooled, deparaffinized, and rehydrated through xylenes and graded ethanol solutions to water. All slides are quenched for 5 minutes in a 3% hydrogen peroxide solution in water to block for endogenous peroxidase. Antigen retrieval can be performed by any suitable technique, e.g., a heat method in which the specimens were placed in a citric acid solution, pH 6.1 (code S1699, Dako, Carpinteria, Calif.) for 20 minutes at 94° C. using a vegetable steamer, then cooled for 15 minutes. Slides are then placed on a Dako Autostainer immunostaining system for use with immunohistochemistry utilizing suitable antibodies. This method is based on the consecutive application of (1) a primary antibody against the antigen to be localized, (2) biotinylated linking antibody, (3) enzyme-conjugated streptavidin, and (4) substrate chromogen (DAB). Slides were then counterstained in Richard-Allan hematoxylin (Kalamazoo, Mich.), dehydrated through graded ethanol solutions, and topped with a coverslip.

A 2-color double immunostain can be performed using any suitable protocol known to those of ordinary skill in the art. For example, without limitation, paraffin-embedded tissue blocks can cut at 4 μm and placed on positively charged slides. Slides with specimens were then placed in a 60° C. oven for 1 hour, cooled, deparaffinized, and rehydrated through xylenes and graded ethanol solutions to water. All slides are then quenched for 5 minutes in a 3% hydrogen peroxide solution in water to block for endogenous peroxidase. Antigen retrieval can be performed using any suitable protocol known to those of ordinary skill in the art. For example, by a heat method in which the specimens were placed in a citric acid solution (pH 6.1) for 25 minutes (as compared with 20 minutes for the individual antibodies mentioned previously) at 94° C. and cooled for 15 minutes using a vegetable steamer. Slides can then, e.g., be placed on a Dako Autostainer immunostaining system, for use with immunhistochemistry.

The first primary antibody is incubated for 30 minutes at room temperature. The detection system, EnVision+ dual link (Dako, code K4061), is incubated for 30 minutes. Lastly, DAB chromogen is added. Before the second primary antibody is applied, serum-free protein block is added (Dako, code X0909) to minimize background and crossover between primary antibodies. The second primary antibody is incubated for 1 hour at room temperature. The EnVision+ dual link (Dako, code K4061) was used again as the detection system and incubated for 30 minutes. NovaRED (Vector Laboratories, Burlingame, Calif.) can be used with second primary so that the staining by the two antibodies can be easily differentiated. Slides are then counterstained in Richard-Allan hematoxylin, dehydrated through graded ethanol solutions, and topped with a coverslip.

Suitable anti-SPARC antibodies can be identified using tissue microarrays to assay for the correct distribution of tumor and fibroblast SPARC staining. Mono and polyclonal antibodies made by standard techniques known in the art can be used.

Tissue microarrays comprising duplicate 0.6-mm cores from the selected blocks can be constructed using a Beecher Instruments Micro Tissue Arrayer. Four-micrometer-thick sections can be cut from completed array blocks and transferred to silanized glass slides. Sections from these arrays then can be stained with hematoxylin and eosin to assess adequacy. Microwave antigen retrieval can consist of placing the slides in 10 mM citrate buffer (pH 6.0) in a pressure cooker (Nordic Ware) and microwaving on high power until the buffer had boiled under pressure for 4 minutes. At this point, microwaving was stopped and the slides were incubated in the pressure cooker for a further 20 minutes, after which they were removed and rinsed. Proteinase antigen retrieval consisted of a 4-minute incubation in protease-1 solution (Ventana) according to the supplier's recommended protocol.

Epitope mapping can also be done using standard techniques known in the art. For example, the protocols from “Epitope Mapping,” Chapter 11, in Using Antibodies by Ed Harlow and David Lane. Cold Spring Harbor Laboratory Press, Cold Spring Harbor, N.Y., USA, 1999, which are hereby incorporated by reference in their entirety. By mapping the epitopes, epitope-specific antibodies can be readily generated by standard techniques.

A SPARC Microenvironment Signature (SMS) can be determined by immunostaining histologic sections of a tumor with a first anti-SPARC antibody, wherein the first anti-SPARC antibody preferentially stains SPARC in tumor cells and with a second anti-SPARC antibody, wherein the second anti-SPARC antibody preferentially stains SPARC in fibroblasts. Seven components of SPARC expression were determined with the two different antibodies: tumor cells, fibroblasts, inflammatory cells, acellular stroma/matrix (stroma), blood vessels, nerves and the other normal anatomy within the tumor. The percent of cells stained in each field, the intensity of staining (0-4) and an score (dependant variable) for each of the components of the tumor were determined (total variables per patient: 7 components×2 antibodies×3 scores=42 variables scored.)

The scoring combined the percent positive cells and staining intensity. The score was negative if no cells or none of the component stained positive. The score was “weakly positive” if <10% of the cells were positive whatever the intensity of staining, the intensity was 2+ or less and <20% of the cells were positive, or the intensity was 1+ and <30% of the cells were positive. The score was “moderately positive” if the intensity was 4+ and 10-40% of the cells were positive, the intensity was 3+ and 10-50% of the cells were positive, the intensity was 2+ and 20-70% of the cells were positive, or the intensity was 4+ or less and 10-40% of the cells were positive or the intensity was 1+ or less and >30% of the cells were positive. The score was “strongly positive” if the intensity was 4+ and >40% of the cells were positive, or the intensity was 3+ and >50% of the cells were positive, the intensity was 2+ and >70% of the cells were positive.

This data was mined using the clustering programs in the Elementspring™ software suite and Nexus™ array analysis programs. In addition, ANOVA or t-test (unpaired) statistics were determined for parameters that clustering suggested had discriminating power for various outcome parameters.

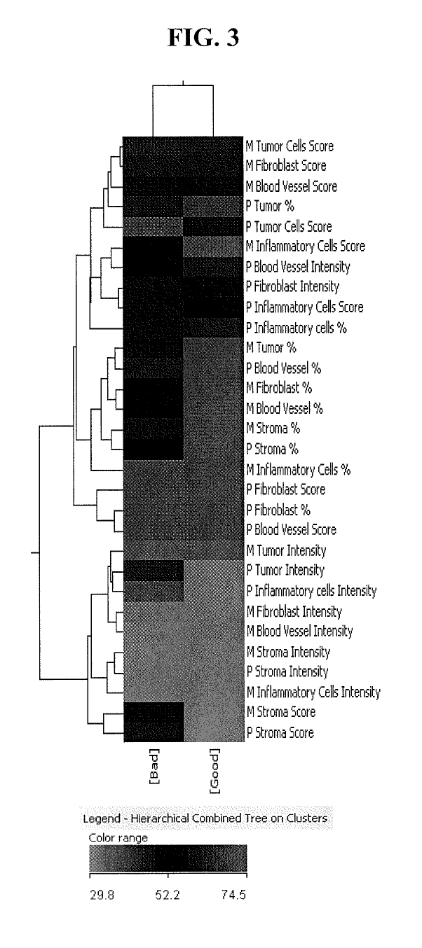

Hierarchical clustering is an extensively used data mining technique which provides a good ‘first pass’ analysis of data. It involves using one of several techniques iteratively, starting with one datapoint (i.e., measured parameter value) or “element,” and combining elements with their nearest neighbor, gradually building clusters and associations of clusters. The final result is a hierarchical tree (e.g., FIG. 3). Distance between clusters is defined by the distance between their average expression patterns. A visual representation of the clusters is created in the form of a hierarchical tree, or dendrogram, familiar and easily understood by all biologists. The tree structure makes it easy to visually see how similar the expression patterns are between elements or sets of elements.

Non-hierarchical clustering techniques group N number of elements into K clusters. Two examples are K-Means clustering and Self Organizing Maps. K-means clustering begins with a predefined (K) number of clusters, or “centroids” and involves a three step process. First, elements are randomly assigned to a centroid. Second the mean inter and intra-cluster distances are then calculated. Finally, elements are moved from one cluster to another. Steps two and three are repeated until intra-cluster distance is minimized and inter-cluster distance in maximized, typically resulting in K round shaped clusters. New elements are grouped in the cluster with the nearest centroid. A centroid is the average of all the points in the cluster. K-means clustering excels at clustering elements where the number of groups is known. For example, a dataset containing cancerous and non-cancerous tissues could be analyzed according to K-means clustering to identify 2 groups of genes: those that change with cancer and those that do not.

Self Organizing Maps (SOM) are generated via neural network techniques to iteratively map nodes into n-dimensional “element space.” This technique incorporates prior knowledge because a partial structure is imposed (the number of clusters and dimensionality must be assigned) prior to analysis. Then, random vectors are created and added to each node. Next, the distance between the vectors and a randomly selected gene are calculated. The vector closest to the gene is updated, making it more like the element's vector. The process is repeated thousands of times until no more changes can be made. This process converts large dimensional element space into something more manageable and understandable.

By the SPARC Microenvironmental Signature (SMS) it meant the pattern of staining with two anti-SPARC antibodies as indicated by the histologic location, intensity, and frequency of immunostaining with each antibody. By “clustering” it is meant the use of any suitable clustering method to group SMSs based on their clinical outcomes and identify the SMS components that contribute to distinguishing one group from another. Suitable methods include, e.g., K-means, Self Organizing Maps and Hierarchical clustering (all of which can be performed by commercially available software known to those of ordinary skill.) A “centroid” is the range of parameters that defines a cluster group. In this application in refers specifically to the SMS component values which distinguish different SMS groups, e.g., the criteria for being classified as a “responder.” Assignment to an outcome group is the process of determining which centroid best represents the data available that defines your group.

While all clustering techniques excel under certain conditions they also have limitations, which will be know to those skilled in the art. For example, hierarchical clustering imposes a rigid relational structure on the data which may or may not reflect reality. K-means clustering and SOM generation require a predetermined number of clusters. This works well in certain situations, but for blind, exploratory data analysis, like determining gene relationships, the proper number of clusters cannot be determined ahead of time. K-means clustering has an additional limitation in that it produces fairly round clusters, resulting in inaccurate identification of close or geometrically shaped clusters. Lastly, although clustering shows an association between groups of elements, no conclusions can be drawn about relationships between elements within a cluster, such as a direction of action.

Reducing the number of elements is an important step which is desirably performed before the above described classification methods can be applied. This should be done so as to preserve as much discriminant information as possible to improve the learning accuracy. Properly defined elements should have the same expression pattern for all samples of the same class, and have different expression patterns from samples belonging to different classes. The “nearest shrunken centroid” method for class prediction uses “shrunken” centroids as prototypes for each class and identifies subsets of elements that best characterize each class. the method “shrinks” each of the class centroids toward the overall centroid for all classes by comparison to a threshold value. This shrinkage makes the classification more accurate by eliminating the effect of noisy elements and as a result automatically selects elements. The element profile of a new sample is compared to each of these class centroids. The class whose centroid that it is closest to, in squared distance, is the predicted class for that new sample.

There are two factors to consider in selecting proper elements for classifications: the distance within a class and the distance between classes. When element levels for all samples in the same class are fairly consistent with a small variance, but are largely different among samples of different classes, the element is considered a good candidate for classification. The difference between a class centroid and the overall centroid for an element is divided by the variance within each class to give a greater weight to elements with lower variance. A threshold value is applied to the resulting normalized class centroid differences. If it is small for all classes, it is set to zero, meaning the element is eliminated. This reduces the number of elements that are used in the final predictive model.

Association rules can be used to identify the relationships between elements, relationships between a gene and several other groups of elements, and ultimately may indicate a particular treatment action. The first step is to discretize the data and convert it to a Boolean or tertiary notation. Then a cut-off value is established relative to which data is categorized as up regulated or down regulated. Up regulated genes, with values higher than the cut-off value, are assigned a value of ‘1.’ Down regulated genes, with values below the cut-off value, are assigned a value of ‘0’. Alternatively, two cut-off values could be assigned, and genes could be categorized as up regulated (and assigned the value of 1), down regulated (and assigned the value of −1) or unchanged (and assigned the value of 0).

Any suitable dose of angiogenesis inhibitor may be used, e.g., Avastin administered at a dose of from about 5 mg/kg to about 15 mg/kg with a dosing cycle of at least 1 week.

Hydrophobic chemotherapeutic agents have an HLB (HLB is hydrophilic/lipophilic balance number) of 1.0 or less, preferably 2.0 or less, most preferably 5.0 or less, and include, e.g. the agents epothilone, docetaxel, paclitaxel. Microtubule inhibitor such as taxanes include epothilone, docetaxel, paclitaxel, and combinations thereof. “Combinations thereof” refers to both the administration of dosage forms including more than one drug, for example, docetaxel and paclitaxel, as well as the sequential but, temporally distinct, administration of epothilone, docetaxel and paclitaxel (e.g., the use of docetaxel in one cycle and paclitaxel in the next). Particularly preferred chemotherapeutic agents comprise particles of protein-bound drug, including but not limited to, wherein the protein making up the protein-bound drug particles comprises albumin including wherein more than 50% of the chemotherapeutic agent is in nanoparticle form. Most preferably the chemotherapeutic agent comprises particles of albumin-bound paclitaxel, such as, e.g., Abraxane™. Suitable nanoparticle formulations are not limited to those that comprise at least about 50% of the active agent in nanoparticle form. Other suitable nanoparticle formulations comprise at least about 60%, preferably at least about 70%, more preferably at least about 80%, or even more preferably at least about 90% of the active agent in nanoparticle form. Moreover, such nanoparticle formulations can most preferably comprise at least about 95% to at least about 98% of the active agent in nanoparticle form.

Suitable therapies for Her2 positive breast cancer also include regimens comprising six cycles of: neoadjuvant nab-Paclitaxel at 125 mg/m² on days 1, 8, 15 of each 28 day cycle, carboplatin AUC6 on day 1 of each 28 day cycle; Trastuzumab with a 4 mg/kg load followed by 2 mg/kg/wk, and Bevacizumab at 5 mg/kg/wk; followed by surgical removal of the primary tumor; and post-operative therapeutically effective amounts of Trastuzumab and Bevacizumab for 52 weeks. Suitable therapies for Her2 negative breast cancer include, e.g., preoperative therapy comprising 6 cycles of 14 days with nab-Paclitaxel (175 mg/m²), gemcitabine (2000 mg/m²), and epirubicin (50 mg/m²); followed by surgical removal; and postoperative therapy comprising (4 cycles of 14 days) and nab-Paclitaxel (220 mg/m²)+gemcitabine (2000 mg/m²).

The following examples further illustrate the invention but, of course, should not be construed as in any way limiting its scope.

### Example 1

The purpose of this study was to evaluate which SPARC isoforms and functions in the tumor microenvironement are responsible for patient outcomes and, in particular, to determine if there were correlations between patterns of SPARC immunostaining and patient outcomes with a nanoparticulate albumin-bound (nab) paclitaxel (i.e., Abraxane®).

nab-Paclitaxel can utilize endogenous pathways of albumin transport to enter tumor cells, including endothelial cell gp60-albumin receptor transport and binding to SPARC secreted by tumors. Initial preclinical studies and a small retrospective clinical study in head and neck cancer suggested that increased endogenous SPARC in tumor tissue may predict a favorable response to nab-paclitaxel treatment (Desai et al. 2009, Trans One. 2, 59-64).

Four prospective studies examined if SPARC tumor immunostaining patterns, i.e., the “SPARC microenvironment signatures” (SMS), could discriminate patients with low and high risks of recurrence when treated with nab-paclitaxel regimens

The outcome of patients from the four clinical trials were evaluated (Table 1).

TABLE 1
Clinical Trials That Provided Specimens and Outcome Data

| | | | | No. Pts with | |
|---|---|---|---|---|---|
| | | | | SPARC | |
| Study | Indication | Phase | No. Pts | IHC | Regimen |
| N057E | Unresectable | II | 76 | 40 | nab-paclitaxel (100-150 mg/m²) |
| | Stage IV | | | | wkly ¾ |
| | Melanoma | | | | carboplatin (AUC 2) wkly |
| | | | | | ¾ |
| CA040 | Metastatic | I/II | 63 | 37 | nab-paclitaxel (100-150 mg/m²) |
| | Pancreatic | | | | wkly ¾ |
| | Cancer | | | | gemcitabine (1000 mg/m2) |
| | | | | | wkly ¾ |
| BRE73 | Neoadjuvant | II | 123 | 83 | Preoperative: |
| | Breast Cancer | | | | (6 cycles of 14 days) |
| | (HER2−) | | | | nab-paclitaxel (175 mg/m²) + |
| | | | | | gemcitabine |
| | | | | | (2000 mg/m2) + epirubicin |
| | | | | | (50 mg/m²) |
| | | | | | Postoperative: (4 cycles of |
| | | | | | 14 days) |
| | | | | | nab-paclitaxel (220 mg/m²) + |
| | | | | | gemcitabine |
| | | | | | (2000 mg/m²) |
| BRE83 | Neoadjuvant | II | 30 | 30 | Preoperative: |
| | Breast Cancer | | | | (6 cycles of 28 days) |
| | (HER2+) | | | | nab-paclitaxel (100 mg/m²) |
| | | | | | wkly ¾ + |
| | | | | | carboplatin (AUC6) + |
| | | | | | trastuzumab (4 mg/kg load, |
| | | | | | then 2 mg/kg/wk) + |
| | | | | | bevacizumab (5 mg/kg/wk) |
| | | | | | Post-operative |
| | | | | | maintenance (1 yr): |
| | | | | | trastuzumab (6 mg/kg) |
| | | | | | q3wk |
| | | | | | bevacizumab (15 mg/kg) |
| | | | | | q3wk |

Overall, this method is based on the consecutive application of (1) a primary antibody against the antigen to be localized, (2) biotinylated linking antibody, (3) enzyme-conjugated streptavidin, and (4) substrate chromogen (DAB). Slides are then counterstained in Richard-Allan hematoxylin (Kalamazoo, Mich.), dehydrated through graded ethanol solutions, and topped with a coverslip. All slides were stained using automated staining equipment (Dako Cytomation Autostainer, Dako, Carpinteria, Calif.).

The immunostaining in this example was performed as described below. A series of antibodies were evaluated against SPARC. Detailed immunohistologic evaluation was performed by a pathologist certified by the American Board of Pathology. Staining scores were assigned on scale of 0-4+, 4+ being the most positive. As it was not known which components of the tumor are important for SPARC's activity, a breakdown of the various components was performed, including staining in the tumor, blood vessels, fibroblasts, stromal cells, inflammatory cells, and the normal anatomy.

Tissue cores from formalin-fixed, paraffin-embedded tumor blocks (2 cores from the most representative areas per block) are arrayed (Beecher Instruments, Silver Spring, Md.) to create a tissue microarray of cores measuring 2.0 mm each and are placed on positively charged slides. Slides with specimens are placed in a 60° C. oven for 1 hour, cooled, deparaffinized, and rehydrated through xylenes and graded ethanol solutions to water. All slides are quenched for 5 minutes in a 3% hydrogen peroxide solution in water to block for endogenous peroxidase.

Antigen retrieval is performed if no staining is seen and with the staining of normal tissue in the same field serving as an internal positive control. Antigen retrieval is performed by a heat method in which the specimens are placed in a citric acid solution, pH 6.1 (code S1699, Dako, Carpinteria, Calif.) for 20 minutes at 94° C. using a vegetable steamer, then cooled for 15 minutes. Slides are then placed on an immunostaining system such as the Dako Cytomation Autostainer (Dako, Carpinteria, Calif.) for use with immunohistochemistry utilizing suitable antibodies.

Two antibodies with differential affinity for SPARC were identified for this study, a monoclonal antibody (indicated hereinafter by “M”) (SPARC monoclonal antibody (R&D Systems, Minneapolis, Minn.), catalog #MAB941 Lot # ECH045011 diluted 1:100 in a tris based diluent) and a polyclonal antibody (indicated hereinafter by “P”) (SPARC polyclonal antibody (R&D Systems, Minneapolis, Minn.), catalog #AF941 Lot # EWN04 diluted 1:50 in a tris based diluents). Histologic sections of tumors were prepared on slides and stained using a standard immunostaining protocol. Briefly, tissue cores from formalin-fixed, paraffin-embedded tumor blocks (2 cores from the most representative areas per block) were arrayed (Beecher Instruments, Silver Spring, Md.) to create a tissue microarray of cores measuring 2.0 mm each and were placed on positively charged slides. Slides with specimens were then placed in a 60° C. oven for 1 hour, cooled, deparaffinized, and rehydrated through xylenes and graded ethanol solutions to water. All slides were then quenched for 5 minutes in a 3% hydrogen peroxide solution in water to block for endogenous peroxidase. Antigen retrieval is performed by a heat method in which the specimens are placed in a citric acid solution (pH 6.1) for 25 minutes (as compared with 20 minutes for the individual antibodies mentioned previously) at 94° C. and cooled for 15 minutes using a vegetable steamer. Slides are then placed on an immunostaining system (Dako, Carpinteria, Calif.), for use with immunhistochemistry.

All slides were quenched for 5 minutes in a 3% hydrogen peroxide solution in water to block for endogenous peroxidase. After a buffer rinse, slides were incubated with antibody M or a negative control reagent for 30 minutes. A mouse horseradish peroxidase polymer kit (Mouse MACH 3 HRP Polymer Kit, Biocare Medical, Concord, Calif.) was incubated for 20 minutes per reagent. After another buffer rinse, DAB chromogen (Dako, Carpinteria, Calif.) was applied for 10 minutes. Hematoxylin was used to counterstain the slides. The same protocol was used for immunostaining specimens with antibody P, although an avidin-biotin detection kit (Biocare Medical, Concord, Calif.), incubated for 15 minutes per reagent, was used in place of the HRP detection kit.

Detailed pathological evaluation of SPARC expression in a series of tumors was performed by a board certified pathologist. The level of SPARC expression, as determined by immunohistochemistry, was scored for different tumor components. Scores were assigned to the level of SPARC expression on scale of 0-3, with 3 being the most positive score, as is commonly done in the art and well known to those of ordinary skill in the art. The monoclonal and polyclonal antibodies used detected different patterns of SPARC expression as shown in Table 2.

TABLE 2
M and P Immunostaining Profiles.

| | Tumor | | | Fibroblast | | |
|---|---|---|---|---|---|---|
| | Anti- | Anti- | | Anti- | Anti- | |
| | body P | body M | | body P | body M | |
| Breast | 30/106 | 35/106 | p = | 82/107 | 26/107 | p < |
| | | | ns | | | 0.0001 |
| Pan- | 20/36 | 7/36 | p = | 18/29 | 5/29 | p = |
| creas | | | 0.0031 | | | 0.0011 |
| Mel- | 30/41 | 20/41 | p = | 19/33 | 14/33 | p = |
| anoma | | | 0.0408 | | | ns |

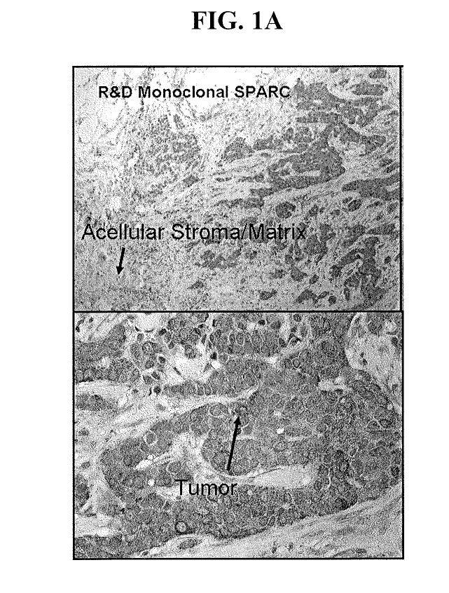

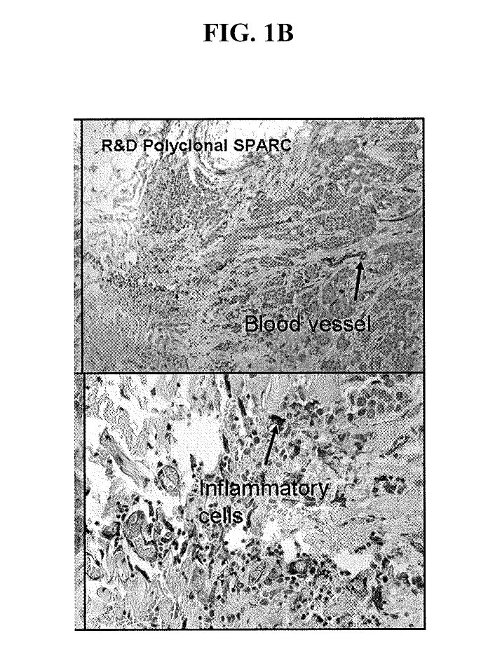

The polyclonal antibody demonstrated preferential staining of fibroblast associated SPARC, while the monoclonal anybody preferential stained tumor associated SPARC. (FIG. 1).

From these staining preferences the following patterns were established analyzed for their predictive value in a series of tumors:

A, when 3+ was found in any of the components.
B, when 3+ was found in any of the components with the monoclonal anti-SPARC antibody.
C, when 3+ was found in any of the components with the monoclonal anti-SPARC antibody.
D, when 3+ was found in tumor cells with both anti-SPARC antibodies.
E, when 3+ was found in fibroblasts with both anti-SPARC antibodies.

Logistic regression and proportional hazard were used to identify any correlations betweens SMS and response, progression free survival (PFS), and overall survival (OS) to SPARC staining pattern in various tumors.

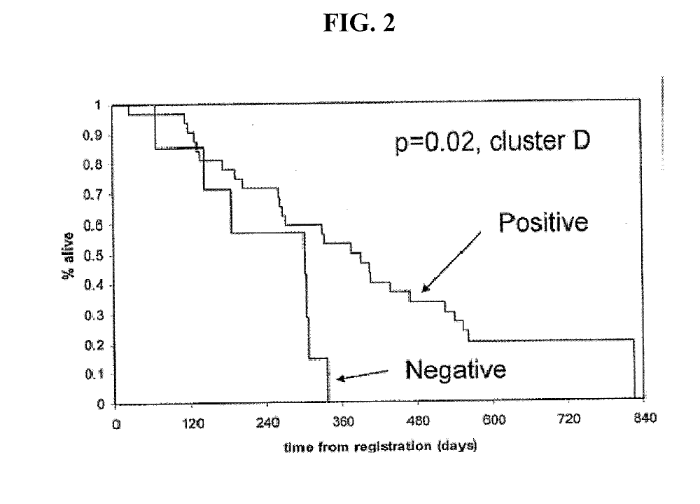

The first tumor set analyzed was a phase II trial of carboplatin and nab-paclitaxel (a.k.a., ABI-007) in patients with unresectable stage IV melanoma (the N057E study). There was a statistically significant correlation between the D pattern and better overall survival (FIG. 2).

The second set of tumors was from patients with advanced pancreatic adenocarcinoma who had been treated with nab-paclitaxel doses (the CA040 study). The 32 patients studied had a full range of responses (Table 3. Response Rates).

TABLE 3
Response Rates

| Response | CR | PR | SD | PD |
|---|---|---|---|---|
| N of 32 pts | 2 | 14 | 14 | 2 |
| | (6%) | (44%) | (44%) | (6%) |

(*CR, Complete Response; PR, Partial Response; SD, No Response and Stable Disease; PD, No Response and Progressive Disease)

Staining of the tumor with the polyclonal antibody was predictive of responsiveness to therapy in this second set of tumors (advanced pancreatic cancer) (one tail t-test, p=0.027). In addition, staining of the tumor cells with the monoclonal antibody predicted a worse overall survival and progression free survival. Further, B pattern staining was predictive of the worst progression free survival with this regimen in these patients with pancreatic adenocarcinoma.

This Example demonstrates that SPARC immunohistochemistry is a fruitful method for predicting response to nab-paclitaxel based chemotherapies.

### Example 2

A more systematic analysis of the staining pattern data from SPARC immunostaining was undertaken to identify patterns which produced prognostic information, Staining pattern data from the same tumor sets studied in Example 1 were mined using various forms of cluster analysis to identify the most distinguishing components of SPARC expression (as indicated by the immunostaining pattern) for response, progression free survival (PFS), and overall survival (OS) to SPARC staining pattern in various tumors. As noted above, the patterns which emerged as prognostically significant are referred to as “SPARC microenvironment signatures” (“SMS”)

SPARC expression was determined with the two different antibodies in seven tumor components: tumor cells, fibroblasts, inflammatory cells, acellular stroma/matrix (stroma), blood vessels, nerves and the other normal anatomy within the tumor. The percent of cells stained, the intensity of staining (0-4) and a “score” was then determined for each of the tumor components. The “score” combined the percent of stained cells and the staining intensity. The score was “negative” if no cells or none of the components stained were positive. The score was “weakly positive” if ≦20% of the cells were positive the intensity was 2+ or less, and also “weakly positive” if ≦30% of the cells were positive or the intensity was 1+ or less. The score was “moderately positive” if the intensity was 4+ and 10-40% of the cells were positive, the intensity was 3+ and 10-50% of the cells were positive, the intensity was 2+ and 20-70% of the cells were positive, or the intensity was 4+ or less and 20-40% of the cells were positive or the intensity was 1+ or less and >30% of the cells were positive. The score was “strongly positive” if the intensity was 4+ and >40% of the cells were positive, or the intensity was 3+ and >50% of the cells were positive, the intensity was 2+ and >70% of the cells were positive.

This data was mined using the clustering programs in the Elementspring® software suite and Nexus® array analysis programs. In addition, ANOVA or t-test (unpaired) statistics were determined for parameters that clustering indicated to have discriminating power.

SMS patterns were identified in the staining data from the BRE 73 breast cancer study. K-Means Cluster Analysis distinguished patients based on immunstaining who had superior PFS. PFS at 24 months was 56% for patients showing “bad” SPARC pattern as opposed to 91% PFS in the patients with a “good” SPARC good pattern.

Moreover, parameters were identified that separated these patients into prognostic groups (Table 4) (“Cut Off Value” is the value required to be classified in the good prognostic group) (see also, FIG. 3).

TABLE 4
SMS Components for PFS in Breast Cancer

| SMS Component | Cut Off Value | p-Value |
|---|---|---|
| P Inflammatory cells % | ≧50% | <0.0001 |
| M Tumor % | ≧70% | <0.0001 |
| P Blood Vessel % | ≧70% | <0.0001 |
| M Fibroblast % | ≧70% | <0.0001 |
| M Blood Vessel % | ≧70% | <0.0001 |
| M Stroma % | ≧70% | <0.0001 |
| P Stroma % | ≧70% | <0.0001 |
| M Inflammatory cells % | ≧70% | <0.0001 |

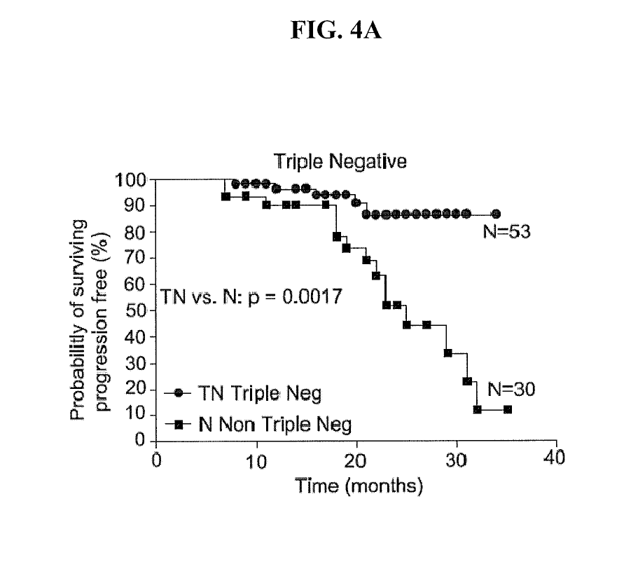

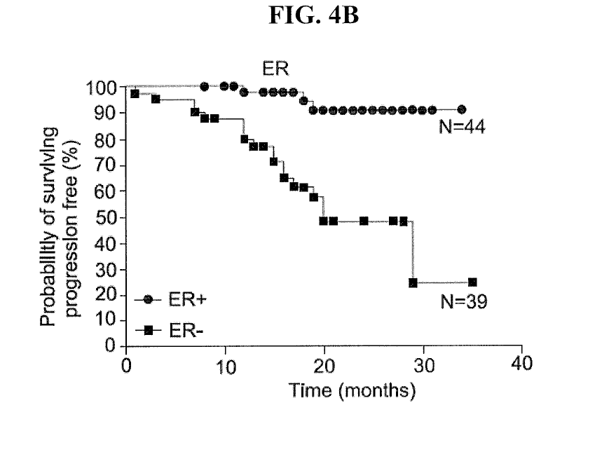

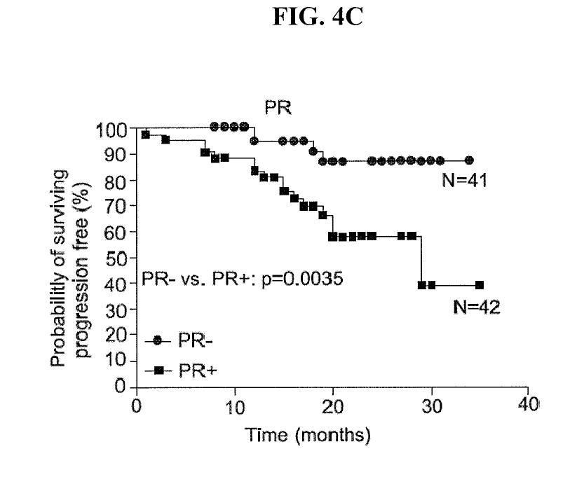

As expected estrogen receptor (ER), progesterone receptor (PR), and Triple Negative (TN) status predicted PFS (FIG. 4A-C). Surprisingly, the SMS functioned as an independent risk factor (i.e., independent of the known risk factors, ER/PR/Triple Negative status (Table 5))

TABLE 5
SMS and Known Risk Factors in Breast Cancer

| | ER- | PR- | Triple Negative |
|---|---|---|---|
| | (N = 39) | (N = 42) | (N = 30) |
| SPARC Good SMS with a | 15/37 (41%) | 16/37 (43%) | 10/37 (27%) |
| known risk factor | | | |
| SPARC Bad SMS with a | 24/46 (52%) | 26/46 (57%) | 20/46 (43%) |
| known risk factor | | | |
| statistics | p = ns | p = ns | p = ns |

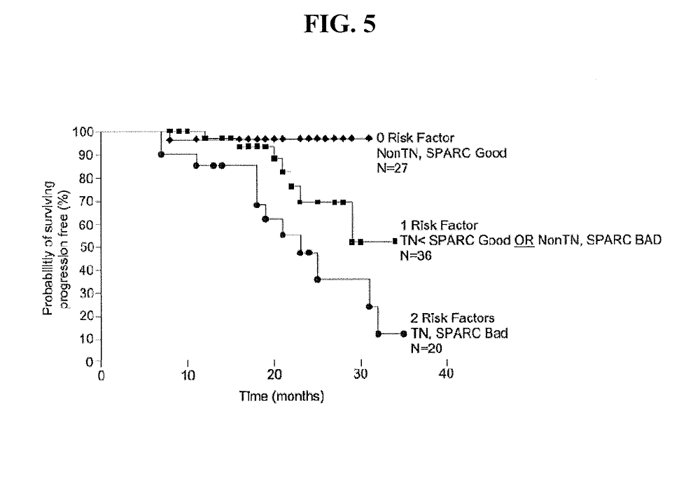

Further, when the SMS was added to known risk factors it improved stratification or discrimination between low and high risk groups based PFS for the nab-paclitaxel based regimen studied (FIG. 5-7). PFS at 24 months was significantly different between groups with 0, 1, and 2 risk factors. But, the addition of SPARC SMS to Triple Negative status further discriminated patients with low risk (0 risk factors) and high risk (>2 risk factors) (log rank p values for different number of risk factors: 0 factors versus 2 factors, p=0.0009; 1 factor vs 2 factors, p=0.039.) (FIG. 5). Also, a group with 1 risk factor was found to be distinct and with intermediate risk.

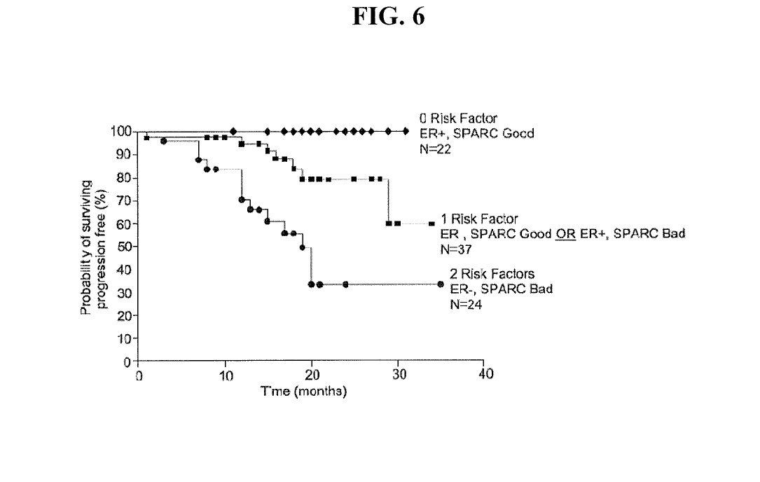

The addition of SPARC SMS clusters to ER further discriminated patients with low risk (0 risk factors) and high risk (2 risk factors) (log rank p values for different number of risk factors: 0 factors vs 2 factors, p=0.0001; 1 factor vs 2 factors, p=0.026.) (FIG. 6).

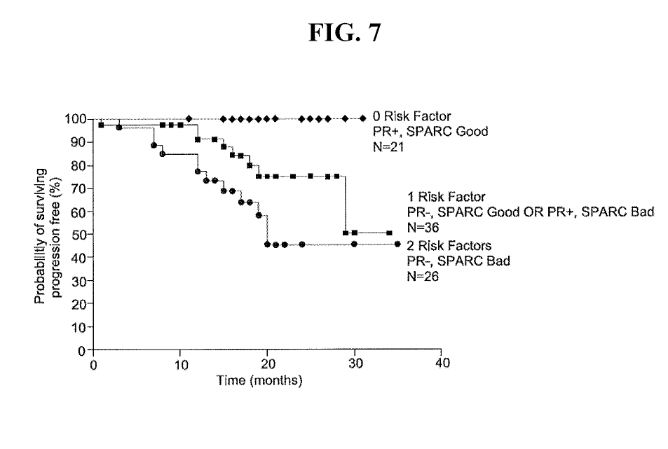

The addition of SPARC SMS clusters to ER further discriminated patients with low risk (0 risk factors) and high risk (2 risk factors) (log rank p values for different number of risk factors: 0 factors versus 2 factors, p=0.0004; 1 factor versus 2 factors, p=ns.) (FIG. 7). These results demonstrate the combination of the SMS with prior art markers of predictive of response to therapy, progression or death can improve the prognostic accuracy of such markers.

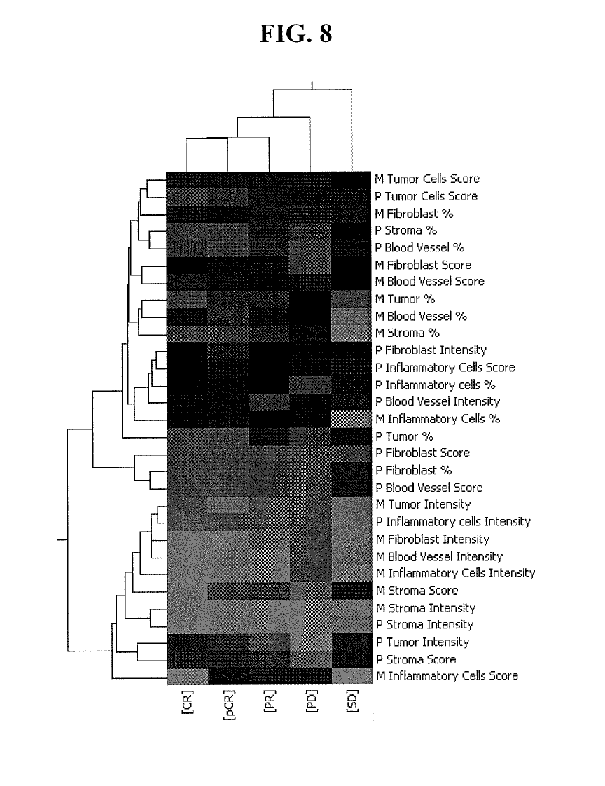

Response was classified as partial complete (pCR), complete response (CR), partial response (PR), (SD), (PD), not available (N/A) (Table 7). The SMS for Response was also identified by cluster analysis (FIG. 8).

TABLE 6
Response Groups

| Response | N |
|---|---|
| pCR | 9 |
| CR | 9 |
| PR | 54 |
| SD | 5 |
| PD | 2 |
| N/A | 4 |

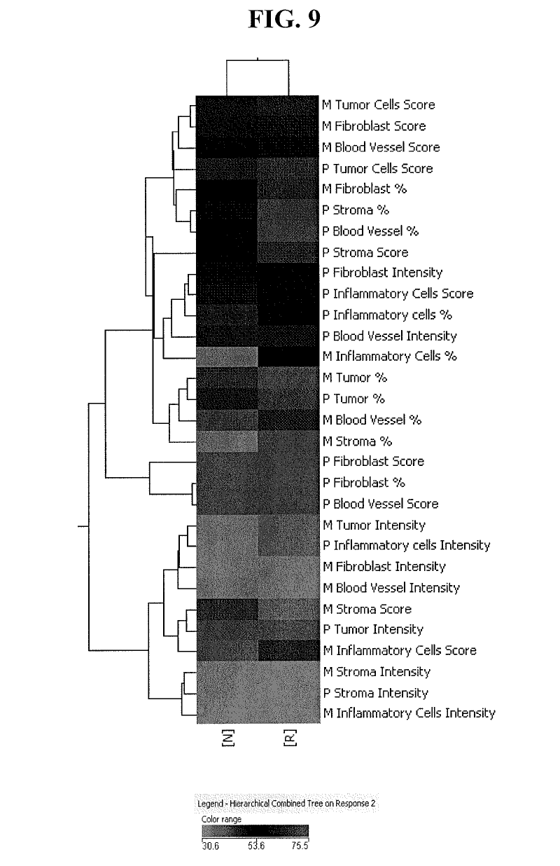

Alternatively, the response outcomes could be shown could be grouped into responders (pCR, CR, PR; n=72) and nonresponders (SD, PD; n=7). For this binary clasification of Response, the SMS was also identified by cluster analysis (FIG. 9).

The parameters involved in the Response SMS were indentified by cluster analysis (FIG. 9) (Table 7).

TABLE 7
Breast Cancer Response SMS Components

| SMS Component | Cut Off Value | p-Value |
|---|---|---|
| M Stroma % | ≧60% | 0.002 |
| M Tumor % | ≧60% | 0.027 |
| M Blood Vessel % | ≧60% | 0.0497 |
| P Tumor % | ≧60% | 0.054 |

Data from the CA040 Pancreatic Cancer Trial were also analyzed. Further the analysis could be extended to cytology specimens from the same patients.

Hierarchical clustering was performed on the pancreatic cancer data so that the patients were divided into two groups based on SMS. These groups were analyzed for PFS and OS outcomes. Clustering reveal that SPARC Low Risk components taken together are significantly higher (˜33%) in SPARC (total score 839 vs 629, sum of significant means) than the High Risk components. Individual components across all the compartments examined (Tumor cell, Fibroblast, Inflammatory cells, Blood Vessels, and Acellular stroma) were higher in SPARC for the Low Risk group.

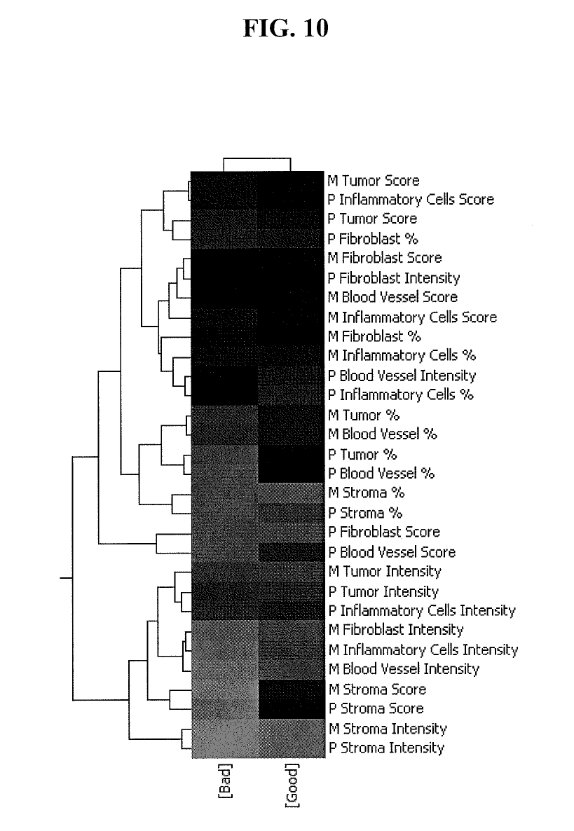

Moreover, using the percent positive cells and quantifying the intensity as 0+=0, 1+=25, 2+=50, 3+=75, 4+=100 and the scores as “negative”=0, “weakly positive”=33, “moderately positive”=66 “strongly positive”=100 gave the following overall results (again, the most important parameters were identified (FIG. 10) (Tables 8) and cut off values (Table 9).

TABLE 8
Pancreatic Cancer SMS Components

| | SPARC | SPARC | |
|---|---|---|---|
| | High | Low | High vs Low Risk |
| Mean of Variable | Risk | Risk | cluster p-value |
| Poly Fibroblast Score | 65.52 | 86.83 | 1.01E−06 |
| Poly Fibroblast Intensity | 40.63 | 67.81 | 1.57E−04 |
| Poly Tumor Intensity | 25.84 | 48.80 | 2.27E−04 |
| Mab Stroma % | 61.88 | 82.00 | 3.28E−03 |
| Poly Inflammatory Cells Intensity | 25.84 | 42.26 | 4.29E−03 |
| Poly Inflammatory Cells Score | 49.05 | 67.14 | 7.49E−03 |
| Poly Blood Vessel % | 50.94 | 68.00 | 8.09E−03 |
| Poly Tumor Score | 54.72 | 75.55 | 8.10E−03 |
| Poly Blood Vessel Intensity | 32.81 | 45.96 | 9.44E−03 |
| Poly Fibroblast % | 54.06 | 70.63 | 1.37E−02 |
| Poly Blood Vessel Score | 63.52 | 75.03 | 2.02E−02 |
| Poly Inflammatory Cells % | 42.66 | 58.50 | 2.81E−02 |
| Poly Stroma Score | 61.88 | 50.55 | 5.00E−02 |
| Mab Stroma Intensity | 21.63 | 16.63 | 8.64E−02 |
| Poly Tumor % | 56.56 | 69.75 | 1.06E−01 |
| Mab Fibroblast Intensity | 25.83 | 32.94 | 1.37E−01 |
| Mab Tumor Intensity | 28.16 | 35.73 | 1.63E−01 |
| Mab Stroma Score | 46.56 | 37.91 | 1.99E−01 |
| Poly Stroma % | 69.06 | 78.00 | 2.51E−01 |
| Mab Blood Vessel Intensity | 22.39 | 24.90 | 4.50E−01 |
| Mab Inflammatory Cells Intensity | 24.31 | 27.03 | 4.54E−01 |
| Mab Fibroblast Score | 56.77 | 53.00 | 5.58E−01 |
| Mab Blood Vessel Score | 52.20 | 48.94 | 6.09E−01 |
| Mab Fibroblast % | 55.78 | 52.38 | 6.31E−01 |
| Mab Tumor % | 65.63 | 63.00 | 7.30E−01 |
| Poly Stroma Intensity | 26.56 | 25.85 | 8.60E−01 |
| Mab Tumor Score | 58.88 | 59.99 | 8.74E−01 |
| Mab Inflammatory Cells Score | 47.53 | 46.81 | 9.04E−01 |
| Mab Blood Vessel % | 59.06 | 58.25 | 9.05E−01 |
| Mab Inflammatory Cells % | 46.88 | 47.38 | 9.47E−01 |
| Sum of all means | 1393.11 | 1617.51 | |

Thus, the cut off values were determined to be those presented in Table 9.

TABLE 9
Pancreatic Cut Offs (using components with significant p-values)

| | Cut Off Value | Cut Off Value |
|---|---|---|
| Component | High Risk | Low Risk |
| P Fibroblast Score | ≦66 | ≧87 |
| P Fibroblast Intensity | ≦41 | ≧68 |
| P Tumor Intensity | ≦26 | ≧49 |
| M Stroma % | ≦49 | ≧82 |
| P Inflammatory Cells Intensity | 51 | 42 |
| P Inflammatory Cells Score | ≦55 | ≧67 |
| P Blood Vessle % | ≦33 | ≧68 |
| P Tumor Score | ≦54 | ≧76 |
| P Blood Vessel Intensity | 64 | 46 |
| P Fibroblast % | ≦54 | ≧71 |
| P Blood Vessel Intensity | ≦64 | ≧75 |
| P Inflammatory Cells % | ≦43 | ≧59 |
| P Stroma Score | ≦62 | ≧51 |

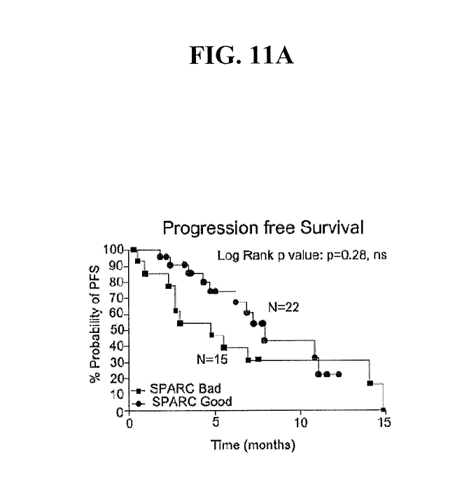

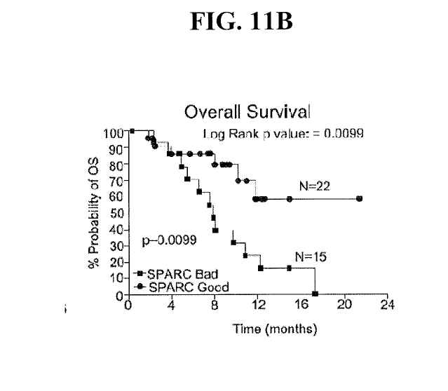

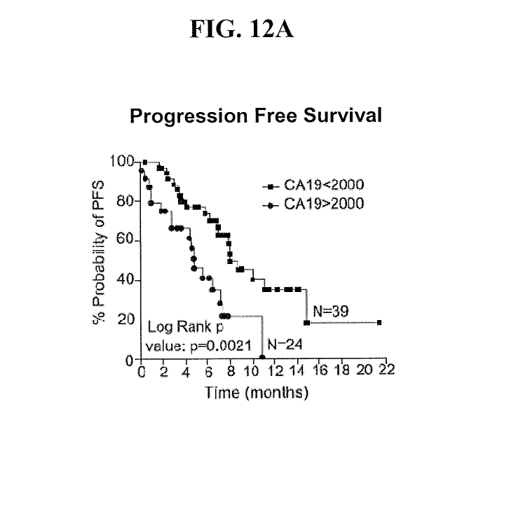

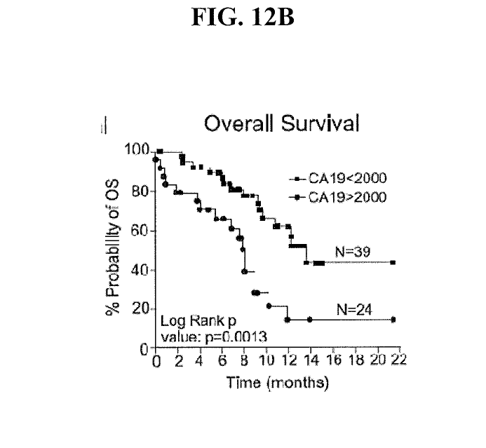

The SMS could distinguish good outcome from bad for OS, but not PFS (FIGS. 11A and B). CA19-9 level is a known risk factor for rapid progression in pancreatic cancer and in the trial CA19-9 level was able to separate PFS and OS groups (FIG. 12). However, there was no correlation between the risk factors SPARC Bad and CA19-9≧2000 U/ml. Accordingly, SPARC and CA 19-1 were found to be independent prognostic factors for overall survival (Table 10).

TABLE 10
SMS for Pancreatic Cancer Is Independent of CA19-9

| | CA19-9 | CA19-9 | |
|---|---|---|---|
| | <2000 U/ml | ≧2000 U/ml | statistics |
| Distribution of Pts | 7/20 (35%) | 6/15 (40%) | p = ns |
| with SPARC Bad | | | |
| signature in CA19-9 | | | |
| groups | | | |
| | | SPARC | |
| | SPARC Bad | Good | statistics |
| Distribution of Pts | 6/13 (46%) | 9/22 (41%) | p = ns |
| with CA19-9 | | | |
| ≧2000 U/ml | | | |
| in SPARC | | | |
| clusters | | | |

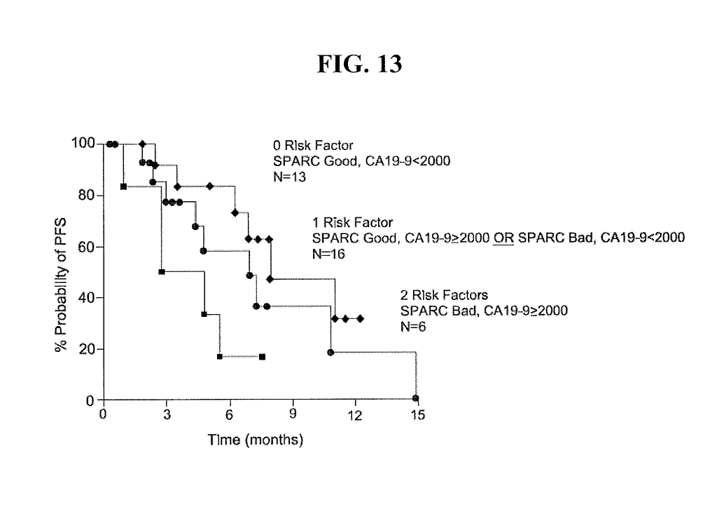

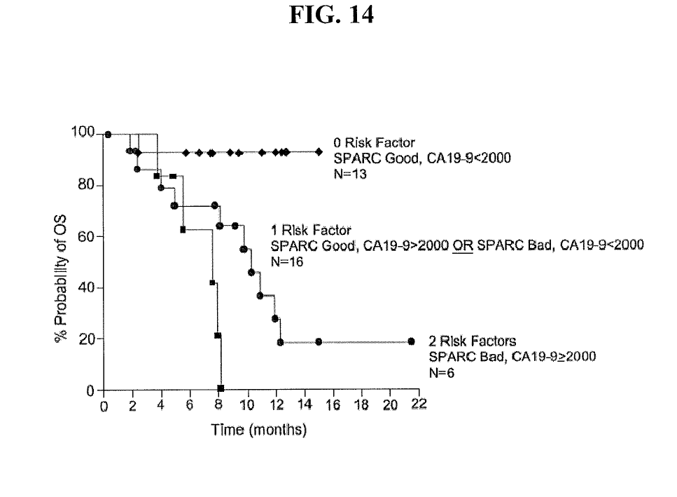

Surprisingly, SMS combined with CA 19-1 level improved stratification PFS and OS (FIGS. 13 and 14).

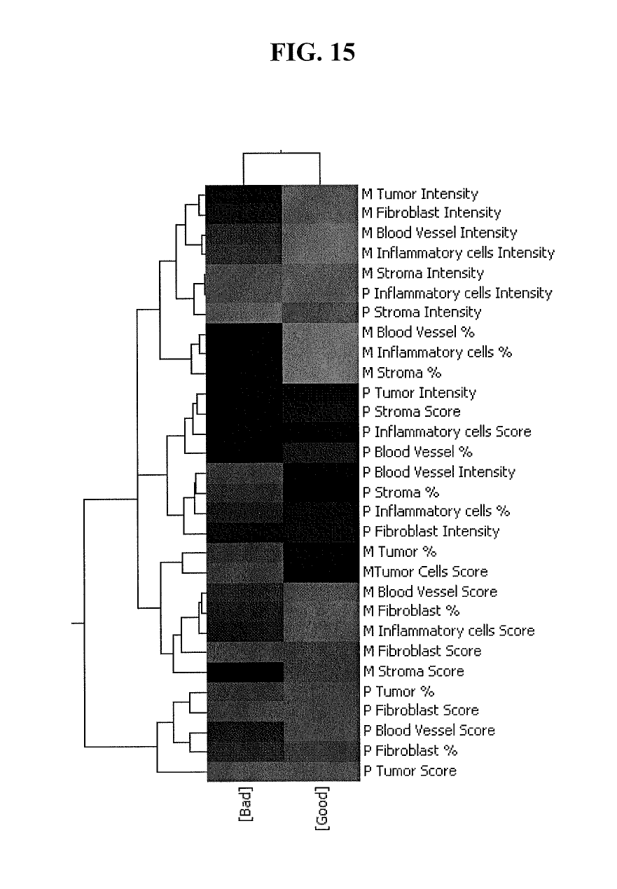

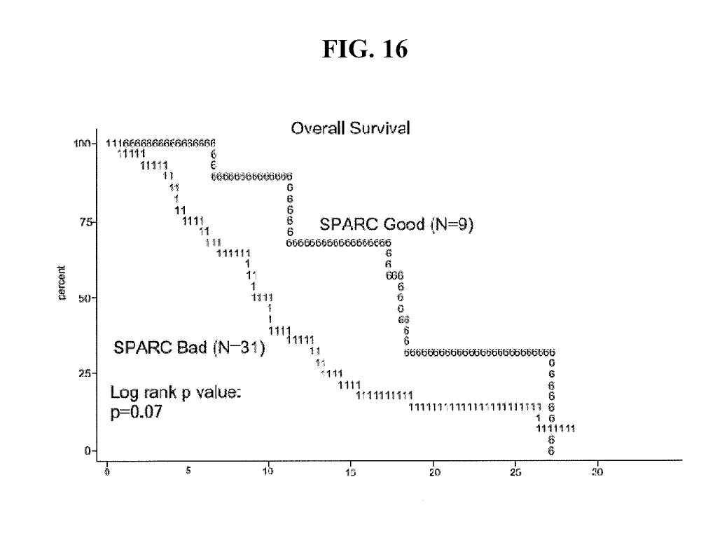

Further analysis of the utility of SMS was undertaken in patients advanced melanoma from the ABX054 Trial. Again, prognostic parameters (Table 11) were identified using hierarchical clustering (FIGS. 15 and 16).

TABLE 11
Melanoma PFS Prognostic Parameters

| SMS Component | Cut Off Value | p value |
|---|---|---|
| M Blood Vessel % | ≦50% | <0.0001 |
| M Stroma Score | slightly positive | <0.0001 |
| M Inflammatory cells % | ≦50% | <0.0001 |
| M Blood Vessel Score | slightly positive | <0.0001 |
| M Inflammatory cells Score | slightly positive | <0.0001 |
| M Stroma % | ≦50% | <0.0001 |
| M Fibroblast Intensity | 1+ to 2+ | <0.0001 |
| M Blood Vessel Intensity | 1+ to 2+ | 0.0006 |
| M Tumor Intensity | 1+ to 2+ | 0.0007 |
| M Tumor Cells Score | slightly positive | 0.0021 |
| M Fibroblast % | ≦50% | 0.0022 |
| M Inflammatory cells Intensity | 1+ to 2+ | 0.0029 |
| M Fibroblast Score | slightly positive | 0.0036 |
| M Tumor % | ≦50% | 0.0205 |

### Example 3

This is an prophetic example of the use of a k-means clustering to generate an SMS and its use to classify individuals into risk groups.

First the centroids for each SMS component must be defined using a training set. Consider a hypothetical data set consisting of the scores of two components of the SMS, e.g., M % tumor and P % Tumor on each of seven individuals:

TABLE 12

| Subject | M % Tumor | P % Tumor |
|---|---|---|
| 1 | 10 | 10 |
| 2 | 15 | 20 |
| 3 | 30 | 40 |
| 4 | 50 | 70 |
| 5 | 35 | 50 |
| 6 | 45 | 50 |
| 7 | 5 | 45 |

This data set is to be grouped into two clusters, e.g., responder and non-responder. As a first step in finding a sensible initial partition, let the M % tumor and P % Tumor values of the two individuals furthest apart (using the Euclidean distance measure) and with known different responses, define the initial cluster means, giving:

TABLE 13

| | | Mean |
|---|---|---|
| | | Vector |
| | Individual | (centroid) |
| Responder | 1 | (10, 10) |
| Cluster | | |
| Nonresponder | 4 | (50, 70) |
| Cluster | | |

The remaining individuals are now examined in sequence and allocated to the cluster to which they are closest, in terms of Euclidean distance to the cluster mean. The mean vector is recalculated each time a new member is added. This leads to the following series of steps:

TABLE 14

| | |
|---|---|
| Responder | Nonresponder |
| Cluster | Cluster |

| | | Mean | | Mean |
|---|---|---|---|---|
| | | Vector | | Vector |
| Step | Individual | (centroid) | Individual | (centroid) |
| 1 | 1 | (10, 10) | 4 | (50, 70) |
| 2 | 1, 2 | (12, 15) | 4 | (50, 70) |
| 3 | 1, 2, 3 | (18, 23) | 4 | (50, 70) |
| 4 | 1, 2, 3 | (18, 23) | 4, 5 | (42, 60) |
| 5 | 1, 2, 3 | (18, 23) | 4, 5, 6 | (43, 57) |
| 6 | 1, 2, 3 | (18, 23) | 4, 5, 6, 7 | (41, 54) |

Now the initial partition has changed, and the two clusters at this stage have the following characteristics:

TABLE 15

| | | Mean |
|---|---|---|
| | | Vector |
| | Individual | (centroid) |
| Responder | 1, 2, 3 | (18, 23) |
| Nonresponder | 4, 5, 6, 7 | (41, 54) |

Next, the mathematical quality of the clusters was verified by comparing each individual's distance to its own cluster mean and to that of the opposite cluster, resulting in:

TABLE 16

| | Distance to | Distance to |
|---|---|---|
| | mean | mean |
| | (centroid) of | (centroid) of |
| | Responder | Nonrespond |
| Individual | Cluster | Cluster |
| 1 | 15 | 54 |
| 2 | 04 | 43 |
| 3 | 21 | 18 |
| 4 | 57 | 18 |
| 5 | 32 | 07 |
| 6 | 38 | 06 |
| 7 | 28 | 11 |

Only individual 3 is nearer to the mean of the opposite cluster than its own. In other words, each individual's distance to its own cluster mean should be smaller that the distance to the other cluster's mean (which is not the case with individual 3). Thus, individual 3 is relocated to the other cluster resulting in the new partition:

TABLE 17

| | | Mean |
|---|---|---|
| | | Vector |
| | Individual | (centroid) |
| Responder | 1, 2 | (13, 15) |
| Nonresponder | 3, 4, 5, 6, 7 | (39, 51) |

This followed by the relocation of individuals based on response, which is again tested mathematically. The iterative relocation would now continue from this new partition until no more relocations occur. However, in this example each individual is now nearer its own cluster mean than that of the other cluster and the iteration stops, choosing the latest partitioning as the final cluster solution.

Any new individuals could then be classified as a responder or a nonresponder based on which centroid they are closer to.

In addition, although two components were used throughout this example, after the training set has been processed, the components which are most discriminative could be determined by any suitable method and only those components used to define the centroids and classify new individuals.

### Example 4

This example describes correlation of plasma SPARC levels with survival. The correlation was determined during a study consisting of two parallel phase II clinical trials (cohorts) to assess the anti-tumor activity and safety profile of the combination of carboplatin and nab-paclitaxel (Abraxane®, also designated ABI-007) in patients with unresectable stage IV malignant melanoma.

Cohort 1 consisted of patients that were previously treated with chemotherapy, and cohort 2 consisted of patients that were newly diagnosed and chemotherapy naïve.

The data presented herein is from a multi-institution cooperative group study conducted through the North Central Cancer Treatment Group (NCCTG). This study was approved by the institutional review boards of all participating institutions. Written informed consent was obtained from all participants. Eligible patients were 18 years of age or older, with unresectable, histologically confirmed, stage IV melanoma. Additional eligibility criteria included a measurable disease as defined by the Response Evaluation Criteria in Solid Tumors (RECIST), an Eastern Cooperative Oncology Group (ECOG) performance status (PS) of 0-2, a life expectancy of 3 months or greater, adequate hematologic and hepatic function, 4 weeks or more elapsed since last chemotherapy treatment (cohort 1 only), radiation therapy, or immunotherapy. Exclusion criteria included: any prior treatment with platinum or taxanes (cohorts 1 & 2), any prior chemotherapy for metastatic disease (cohort 2), active infection, New York Heart Association Class III or IV, peripheral neuropathy of grade 2 or higher; other malignancy in the last 5 years (except for non-melanomatous skin cancer or carcinoma in situ of the cervix) or untreated metastatic melanoma to the brain or progression of brain metastasis within 3 months of study entry. Women who were pregnant or breast feeding were not enrolled.

Eligible patients (both cohorts) were treated with 100 mg/m² of nab-paclitaxel by intravenous infusion over 30 minutes followed by carboplatin (CBDCA) with a target AUC of 2 (by Calvert formula with Cockroft and Gault Equation and actual body weight) over 30 minutes on days 1, 8, and 15 of a 28 day cycle, for a maximum of 8 cycles. If patients did not develop excessive toxicity or progressive disease, treatment beyond 8 cycles was at the discretion of the treating physician. Within 14 days of registration, patients underwent a complete physical exam, assessment of ECOG PS, complete blood cell count (CBC), comprehensive metabolic panel including lactic dehydrogenase (LDH), and a tumor assessment by conventional CT or MRI or spiral CT. Prior to each cycle of treatment, patients underwent a physical exam, toxicity assessments, and blood draws for hematologic and chemistry groups. Tumor status was assessed every 8 weeks until progression using RECIST criteria. On day 1 of each treatment cycle, treatment was withheld if absolute neutrophil count (ANC) was less than 1,500/mm³, platelet count (PLT) was less than 100,000/mm³, the patient developed a grade 2 or higher AST neuropathy, or other grade 3 or higher non-hematologic toxicity. When patients had recovered from these toxicities, treatment was re-started with a 20% dose reduction in both agents. On days 8 or 15 of each treatment cycle, treatment was omitted if: ANC was less than 1,000/mm³ or PLT was less than 100,000/mm³ or patient developed either a grade 2 or higher neuropathy or grade 3 or higher non-hematologic toxicity. Study treatment was terminated if toxicities did not recover to acceptable levels within 4 weeks and/or if patients required a third dose reduction due to toxicity. All patients received standard supportive care, including antiemetics, antibiotics, blood/platelet transfusions, erythropoietin and colony stimulating factors at the discretion of the treating physician.

Thirty five patients were accrued to Cohort 1, and 41 patients were accrued to Cohort 2 between Nov. 15, 2006 and Jul. 31, 2007 (Table 2). In Cohort 1 (PT) 1 patient canceled participation after signing a consent form but prior to the start of treatment. As such, the study Cohort 1 consists of 34 patients (67.6% male) who began study treatment. The median age at enrollment was 60 years (ages ranged from 28 to 84 years). In Cohort 2 (CN), 2 patients canceled participation after signing a consent form but prior to the start of treatment. As such, the study Cohort 2 consists of 39 patients (59.0% male) who began study treatment. The median age at enrollment was 59 years (ages ranged from 23 to 91 years).

For Cohort 1 the median number of cycles administered was 4 cycles (total: 135 cycles, range: 1-10). Twenty one patients (61.8%) were omitted from treatments on day 8 or 15 of treatment or had at least one dose reduction. This was primarily due to severe neutropenia, fatigue, and neuropathy. The main reason for study discontinuation was progression of disease (27 patients).

For Cohort 2, the median number of cycles administered was 4 cycles (total: 193 cycles, range: 1-25). Twenty five patients were omitted from treatements on day 8 or 15 of treatment or had at least one dose reduction, largely due to severe neutropenia and neuropathy. The primary reason for study discontinuation was progression of disease (27 patients).

The prognostic utility of plasma SPARC was evaluated by stratifying patients into “high” and “low” SPARC groups. As the median for plasma SPARC was 431 ng/ml, high SPARC group was defined as patients with plasma SPARC above 431 ng/ml. The breakdown of the patient population is shown in Table 18. As shown in Table 18, with one exception, the results show that “high SPARC” patients tend to have worse progression free survival (PFS) and overall survival (OS) than their “low SPARC” counterparts, although only OS in the prior chemotherapy group was found to be statistically significant (p=0.01).

TABLE 18

| | Median | P-value | N |
|---|---|---|---|
| Progression Free Survival | | | |
| Prior Chemotherapy group | | 0.21 | 31 |
| Low SPARC | 141 days | | 17 |
| High SPARC | 58 days | | 14 |
| No Prior Chemotherapy group | | 0.47 | 35 |
| Low SPARC | 122 days | | 16 |
| High SPARC | 167 days | | 19 |
| Overall Survival | | | |
| Prior Chemotherapy group | | 0.01 | 31 |
| Low SPARC | 378 days | | 17 |
| High SPARC | 206 days | | 14 |
| No Prior Chemotherapy group | | 0.43 | 35 |
| Low SPARC | 426 days | | 16 |
| High SPARC | 304 days | | 19 |

While the overall response rate differed significantly between the two cohorts (25.6% vs 8.8%), there was no difference in progression free survival or overall survival (FIG. 17 A-B). Overall, treatment was moderately well tolerated with the main toxicities being nausea, vomiting, peripheral neuropathy, and cytopenias (neutropenia, thrombocytopenia, leukopenia).

These results show that low circulating SPARC level was associated with improved overall survival. Additionally, the combination of nab-paclitaxel and carboplatin is a feasible therapeutic option for patients with metastatic melanoma who are either previously treated or chemotherapy naïve.

### Example 5

This example describes the evaluation of plasma SPARC concentration in samples derived from metastatic melanoma patients and healthy individuals.

ELISA plates were coated with 2.5 μg/ml SPARC binding polyclonal antibody (R&D Biosystems, Minneapolis, Minn.) in 50 mM carbonate buffer overnight at 4° C. Plates were washed 4 times with PBS/0.1% Tween 20 (PBST) and blocked for 2 hours at room temperature (RT) with casein blocking/dilution buffer (Thermo Fisher Scientific Inc., IL). For the generation of a SPARC standard curve, known concentrations of human platelet SPARC protein (Hematologic Technologies, Essex junction, VT) was diluted in blocking/dilution buffer containing SPARC negative 10% pooled normal human heparin plasma (PNHP). Before testing, patient samples were diluted 1/10 in blocking/dilution buffer. After removal of the blocking solution and three washes with PBST, standards and diluted plasma samples were plated onto the ELISA plates at 100 μl/well in duplicates and incubated for 2 hours at room temperature (RT), followed by three additional washes with PBST. For detection of bound SPARC, 100 ul of 0.5 μg/mlbiotinylated anti SPARC monoclonal antibody (R&D Biosystems, Minneapolis, Minn.) in blocking/dilution buffer was added and incubated for 1 hour at RT, followed by 3 PBST washes. This was followed by 100 ul/well of 1:20000 diluted Streptavidin-Horseradish peroxidase (HRP) was added and incubated for 1 h at RT. After three PBST washes, 100 ul of HRP-Substrate TMB (KPL #52-00-03) was added to each well and OD at 650 nm was monitored. The reaction was stopped for measurement at OD 0.6 to 0.8 with 2N sulfuric acid. The optical density of the wells was read on an ELISA plate reader (Molecular Devices; Sunnyvale, Calif.) at 450 nm within 30 minutes.

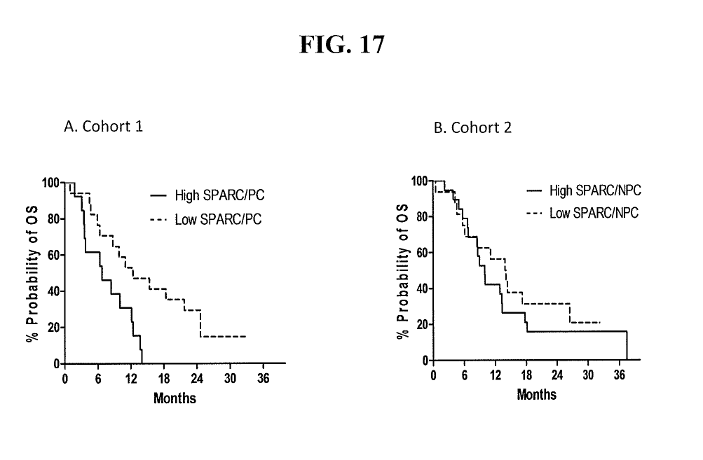

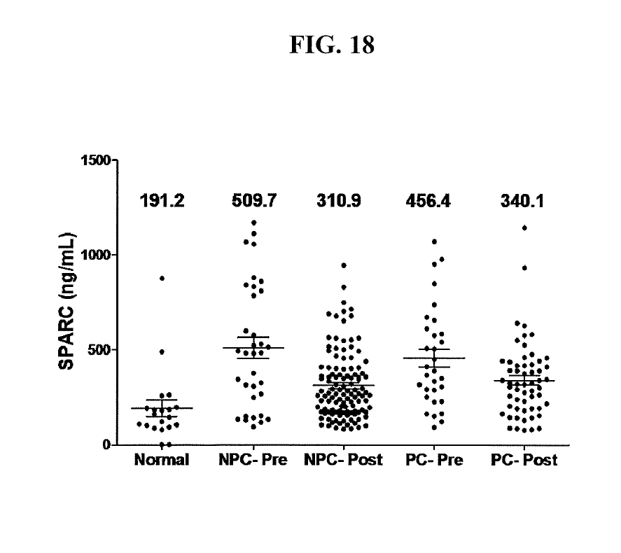

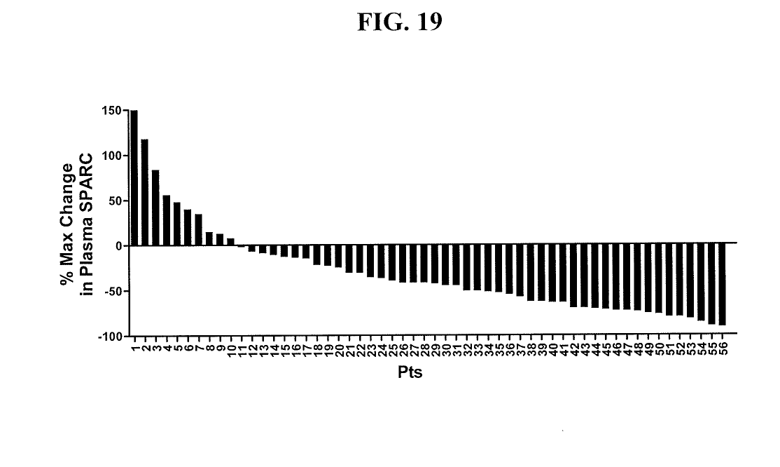

The results from a total of twenty samples derived from healthy individuals were compared to results from 65 cancer patient plasma samples as shown in FIG. 18. As shown in Table 19, analysis of the ELISA results revealed a statistically significant difference in the SPARC concentrations of both groups. SPARC levels in healthy individuals were determined at a median concentration of 192 ng/ml whereas the median plasma SPARC concentration in cancer patient samples was measured at 390 ng/ml (p value 0.0002) (FIG. 18). Additionally, treatment was followed with significant drop in plasma SPARC in the majority of the patients (FIG. 19).

TABLE 19

| | | | P Value (Student's t-test) | |
|---|---|---|---|---|
| | | SPARC | | vs Pre- |
| | N | (ng/mL) | vs Normal | treatment |
| Normal | 20 | 191.2 | — | — |
| No Prior Chemotherapy - | 34 | 509.7 | 0.0002 | — |
| Pre-treatment | | | | |
| No Prior Chemotherapy - | 116 | 310.9 | 0.0068 | <0.0001 |
| Post-treatment | | | | |
| Prior Chemotherapy - | 31 | 456.4 | 0.0003 | — |
| Pre-treatment | | | | |
| Prior Chemotherapy - | 57 | 340.1 | 0.0048 | 0.022 |
| Post-treatment | | | | |

These results demonstrate increased SPARC expression in metastatic melanoma patients and could be positively correlated with tumor burden.

### Example 6

This example demonstrates the preparation of a SPARC microenvironment signature (SMS).

A series of antibodies against SPARC were evaluated for their binding characteristics in a range of normal and tumor tissues. The SPARC expression pattern, as determined by immunostaining, in various components of tumors was determined including the SPARC expression levels in tumor cells, blood vessels, fibroblast, stroma, inflammatory cells, and the adjacent normal tissues. Two antibodies were identified with differential affinity for SPARC and were employed in follow up studies. Specifically, the pattern of staining was determined using a monoclonal antibody (“antibody M”) (SPARC monoclonal antibody (R&D Systems, Minneapolis, Minn.), catalog #MAB941 Lot # ECH045011 diluted 1:100 in a tris based diluent) and a polyclonal antibody (“antibody P”) (SPARC polyclonal antibody (R&D Systems, Minneapolis, Minn., catalog #AF941 Lot # EWN04 diluted 1:50 in a tris based diluents).

Histologic sections of tumors were prepared on slides and stained using a standard immunostaining protocol. Briefly, tissue cores from formalin-fixed, paraffin-embedded tumor blocks (2 cores from the most representative areas per block) were arrayed (Beecher Instruments, Silver Spring, Md.) to create a tissue microarray of cores measuring 2.0 mm each and were placed on positively charged slides. Slides with specimens were then placed in a 60° C. oven for 1 hour, cooled, deparaffinized, and rehydrated through xylenes and graded ethanol solutions to water. All slides were stained using automated staining equipment (Dako Cytomation Autostainer, Dako, Carpinteria, Calif.).

All slides were quenched for 5 minutes in a 3% hydrogen peroxide solution in water to block for endogenous peroxidase. After a buffer rinse, slides were incubated with antibody M or a negative control reagent for 30 minutes. A mouse horseradish peroxidase polymer kit (Mouse MACH 3 HRP Polymer Kit, Biocare Medical, Concord, Calif.) was incubated for 20 minutes per reagent. After another buffer rinse, DAB chromogen (Dako, Carpinteria, Calif.) was applied for 10 minutes. Hematoxylin was used to counterstain the slides. The same protocol was used for immunostaining specimens with antibody P, although an avidin-biotin detection kit (Biocare Medical, Concord, Calif.), incubated for 15 minutes per reagent, was used in place of the HRP detection kit.

Detailed pathological evaluation of SPARC expression in a series of tumors was performed by a board certified pathologist. The level of SPARC expression, as determined by immunohistochemistry, was scored for different tumor components. Scores were assigned to the level of SPARC expression on scale of 0-3, with 3 being the most positive score, as is commonly done in the art and well known to those of ordinary skill in the art.

The polyclonal antibody demonstrated preferential staining of SPARC in fibroblasts. While the monoclonal anybody preferably stained SPARC in tumor cells.

Logistic regression and proportional hazard were used to determine the correlation between response, progression-free survival (“PFS”) and overall survival (“OS”) to the SPARC pattern.

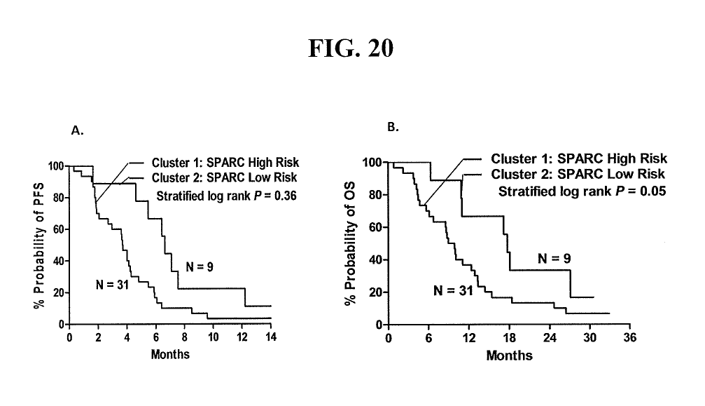

One of the tumor sets was a phase II trial of carboplatin and nab-paclitaxel (ABI-007) in patients with unresectable stage IV melanoma. Specifically, nab-paclitaxel (100 mg/m2) and Carboplatin (AUC2) were administered on days 1, 8, and 15 of a 28 day cycle. SMS of the tumor biopsies were used to group the patients into two clusters, high risk (cluster 1) and low risk (cluster 2). As shown in Table 19 and FIGS. 20A-B, high risk and low risk SPARC signatures were correlated with progression-free survival and overall survival.

TABLE 19

| | Total | Total | Median | % PFS at | Median | % OS at | P |
|---|---|---|---|---|---|---|---|
| | N | Events | PFS (months) | 6 months | OS (months) | 12 months | (Log-rank) |
| Cluster 1 | 31 | 29 | 3.7 | 17% | 9.4 | 37% | 0.0572 |
| (High Risk) | | | | | | | |
| Cluster 2 | 9 | 7 | 6.6 | 67% | 17.7 | 67% | |
| (Low Risk | | | | | | | |

These results show that SPARC microenvironment signature alone can discriminate between low risk and high risk groups with respect to progression free survival and overall survival.

### Example 7

This example describes analysis of the correlation between SPARC microenvironment signature and plasma SPARC levels.

As described in Examples 1-6 above, plasma SPARC levels and SMS were analyzed and the results combined to determine correlations for patient outcomes.

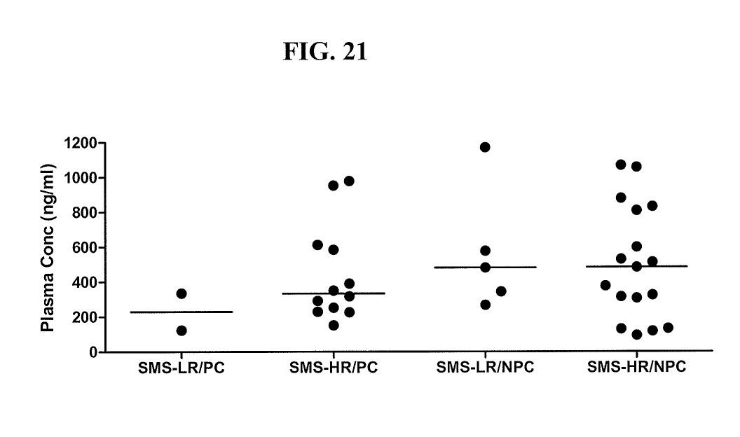

As shown in FIG. 21, baseline plasma SPARC was similar between SMS high-risk and SMS low-risk groups. Patients were coded as having a risk level of 0, 1, or 2, based on baseline plasma SPARC and SMS high risk versus low risk. A risk level of 0 is identified as low baseline plasma SPARC, with SMS low risk. A risk level of 1 is identified as high baseline plasma SPARC or SMS high risk. A risk level of 2 is identified as high baseline plasma SPARC and SMS high risk. Data for overall survival and progression free survival are shown in Table 4.

TABLE 20

| | Median | | Median | |
|---|---|---|---|---|
| | Progression Free | % Progression | Overall | % Overall |
| | Survival | Free Survival | Survival | Survival at 12 |
| | (months) | at 6 months | (months) | months |
| 0 Risk | 6.1 | 50% | 14.1 | 50% |
| 1 Risk | 4.1 | 21% | 14.4 | 53% |
| 2 Risks | 3.6 | 25% | 9.5 | 33% |

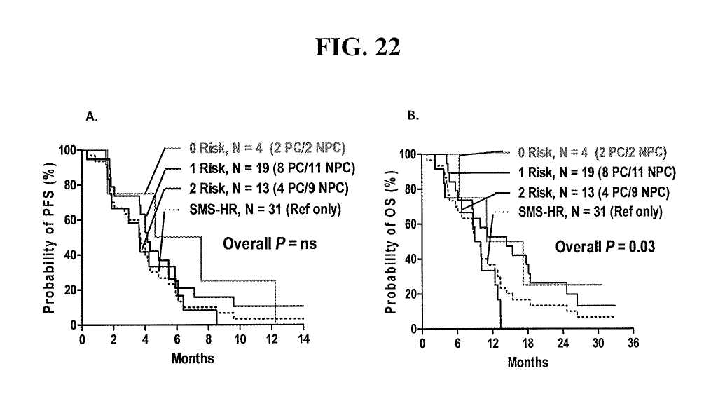

As shown in FIG. 22A-B, there was a general trend to worse progression free survival and overall survival with increasing risk level, although results were not significantly different for progression free survival of patients in the 2 Risks group.

These results show that patients with high plasma SPARC and high-risk SMS had significantly worse overall survival.

### Example 8

This example describes analysis of the correlation between plasma LDH levels and survival.

Baseline plasma LDH levels in 76 Stage IV unresectable melanoma patients were determined using ELISA and correlated with survival rates.

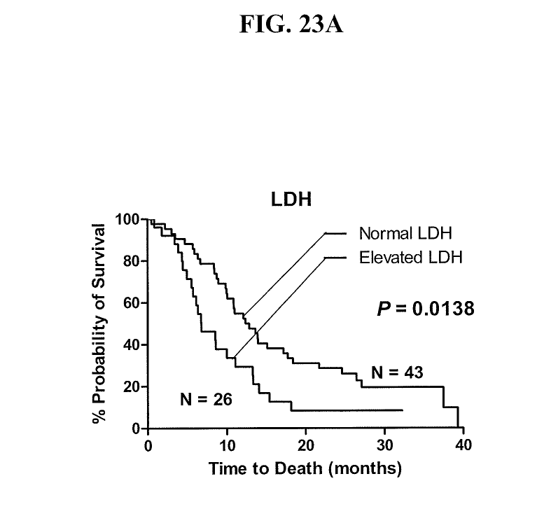

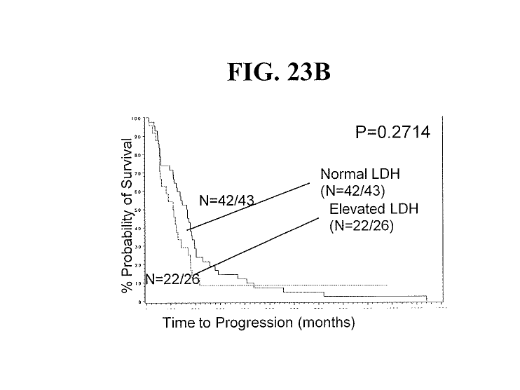

As shown in Table 21 and FIG. 23A (Overall Survival) and 23B (Progression Free Survival), plasma LDH levels were significantly elevated in some melanoma patients, correlating to decreased overall survival as well as decreased progression-free survival. The patients were then treated with nab-paclitaxel (nab-P, 100 mg/m²) and carboplatin (C, AUC 2) on d 1, 8, and 15 of a 28 day cycle until disease progression.

TABLE 21

| | | | Median OS | P (log- |
|---|---|---|---|---|
| LDH | Total N | Total Events | (months) | rank) |
| Normal | 43 | 35 | 12.9 | 0.0138 |
| Elevated | 26 | 22 | 6.8 | |

These results show that plasma LDH alone can predict overall survival of melanoma patients.

### Example 9

This example describes analysis of the correlation between SPARC microenvironment signature and plasma LDH levels.

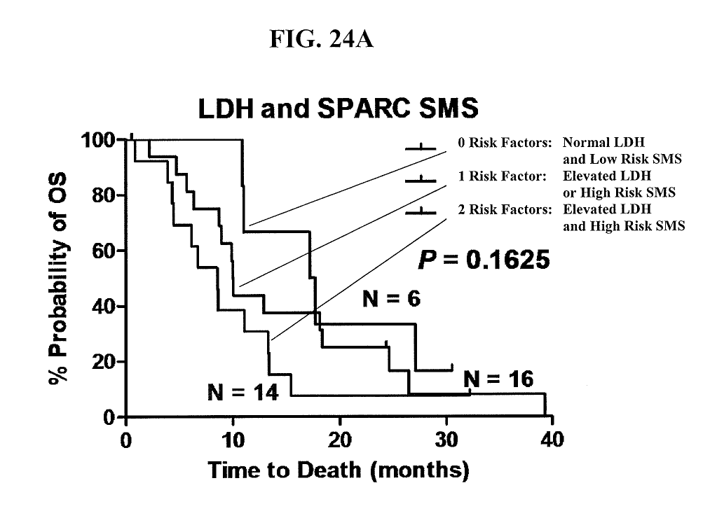

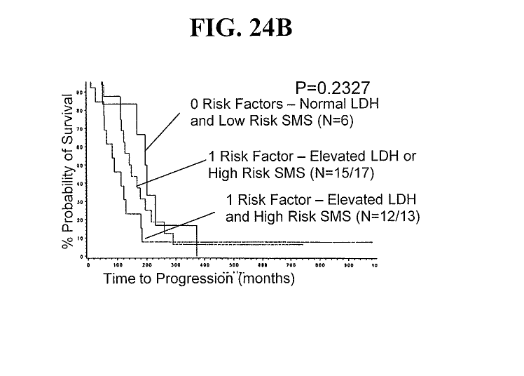

As described in Examples 1-3, 6 and 8 above, plasma LDH levels and SMS were analyzed (Table 22) and the results combined to determine correlations for patient outcomes, as shown in Table 23 and FIG. 24A (Overall Survival) and 24B (Progression Free Survival).

TABLE 22

| | Normal | High | | |
|---|---|---|---|---|
| SMS | LDH | LDH | Total | P |
| High Risk | 6 | 3 | 9 | ns |
| Low Risk | 14 | 13 | 27 | |
| Total | 20 | 16 | 36 | |

TABLE 23

| LDH/SPARC SMS | 0 Risks | 1 Risk | 2 Risks |
|---|---|---|---|
| % Overall Survival at | 67% | 44% | 30% |
| 12 months | | | |
| Median Overall | 17.5 | 10 | 8.6 |
| Survival (months) | | | |

These results show that in melanoma patients, normal LDH and low risk SPARC signature better predicted good overall survival than normal LDH alone.

### Example 9

This example describes analysis of the correlation between plasma SPARC levels and plasma LDH levels.

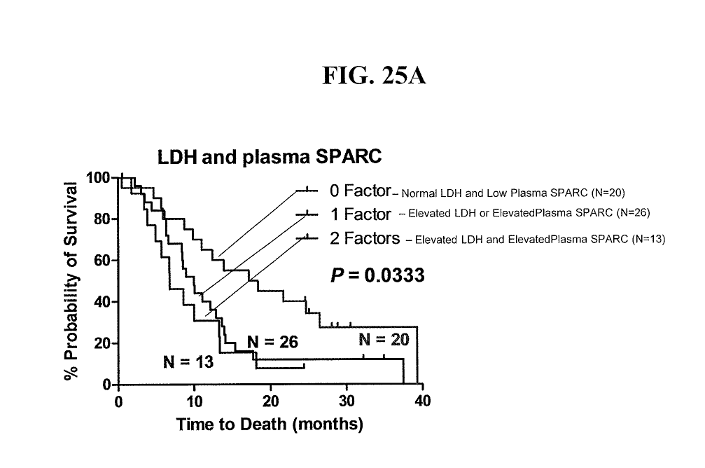

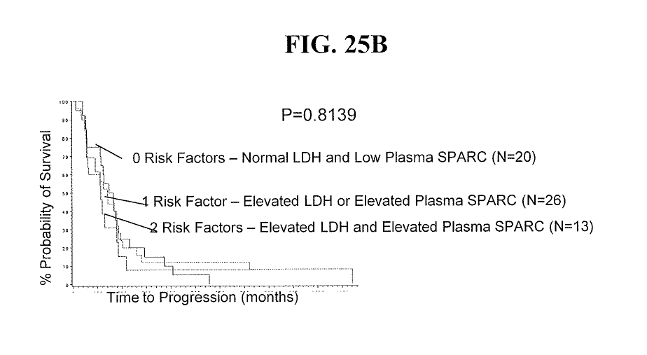

As described in Examples 4-5 and 8 above, plasma SPARC levels and plasma LDH levels were analyzed (Table 24) and the results combined to determine correlations for patient outcomes as shown in Table 25 and FIG. 25A (Overall Survival) and 25B (Progression Free Survival).

TABLE 24

| | Normal | High | | |
|---|---|---|---|---|
| Plasma SPARC | LDH | LDH | Total | P |
| Low <431 ng/mL | 20 | 8 | 28 | ns |
| High >431 ng/mL | 18 | 13 | 31 | |
| Total | 38 | 21 | 59 | |

TABLE 25

| LDH/SPARC SMS | 0 Risks | 1 Risk | 2 Risks |
|---|---|---|---|
| % Overall Survival at | 65% | 40% | 31% |
| 12 months | | | |
| Median Overall | 17.8 | 10 | 6.8 |
| Survival (months) | | | |

These results show that in melanoma patients, normal LDH and low baseline plasma SPARC levels better predicted good overall survival than normal LDH alone.

### Example 10

This example describes the results of a clinical trial investigating the treatment of women with triple-negative metastatic breast cancer (TNMBC) with a novel combination of therapeutics: nab-paclitaxel, carboplatin and bevacizumab.

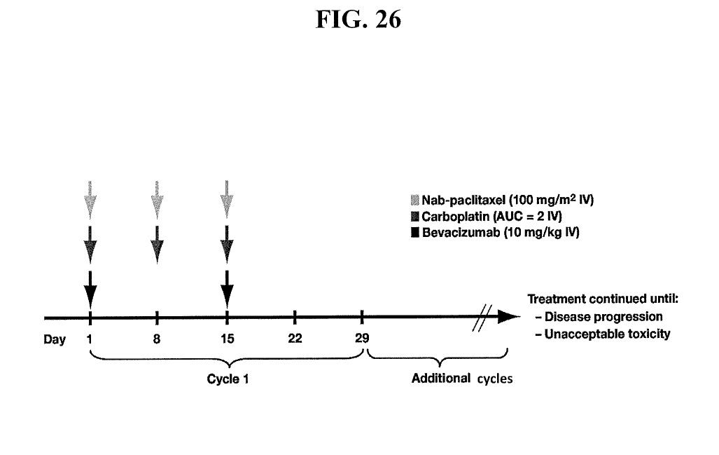

In a phase II, multicenter study, 29 women with newly diagnosed or relapsed TNMBC were treated in 28-day cycles with nab-paclitaxel (100 mg/m²) and carboplatin (AUC=2) administered on days 1, 8 and 15 and Bevacizumab (10 mg/kg) administered on days 1 and 15. Treatment continued according to this schedule until unacceptable toxicity or disease progression was observed. Progression free survival (PFS), objective response rate (ORR), and clinical benefit rate (CBR) were determined. Biopsies of primary tumors and metastatic tumors were taken for subsequent immunohistochemical (IHC) staining and the determination and analysis of SPARC SMS's (see Examples 11 and 12). The treatment regimen for the study is summarized in FIG. 26.

The ORR was 89% (4 patients showing a complete response [CR], 20 patients showing a partial response [PR], 2 patients showing stable disease [SD], 1 patient showing progressive disease [PD], and 2 patients that were non-evaluable). In addition to being effective, this combination was well tolerated, with grade 3/4 toxicities including neutropenia, thrombocytopenia, neuropathy and anemia.

### Example 11

This example demonstrates how the SPARC SMS of biopsies of metastatic tumors correlates with outcomes in the TNMBC patients treated with a combination of nab-paclitaxel/carboplatin/bevacizumab in the study described in the previous example.

For 20 of the patients participating in the study, the SMS in primary and metastatic tumor biopsies was measured using the validated immunohistochemistry (IHC) method described herein (see Example 6). In brief, a board certified pathologist used the two previously described antibodies: one monoclonal and one polyclonal (See Example 6) to measure SPARC expression in seven tumor components: tumor cells, fibroblasts, inflammatory cells, acellular stroma/matrix, blood vessels, nerve tissue, and finally normal tissue within the tumor.

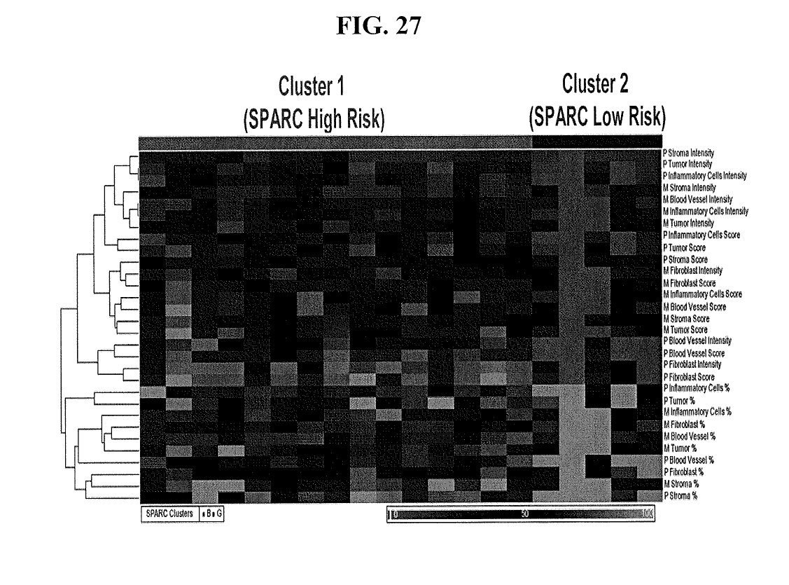

These samples were then scored based three variables: the percentage of cells stained in each field, the intensity of staining, and finally, an overall score (a dependent variable). These data were then analyzed and grouped into clusters according to patient response data (e.g. PFS) using array analysis programs available from Partek (St. Louis, Mo.). A heat diagram displaying the results of this clustering is provided in FIG. 27.

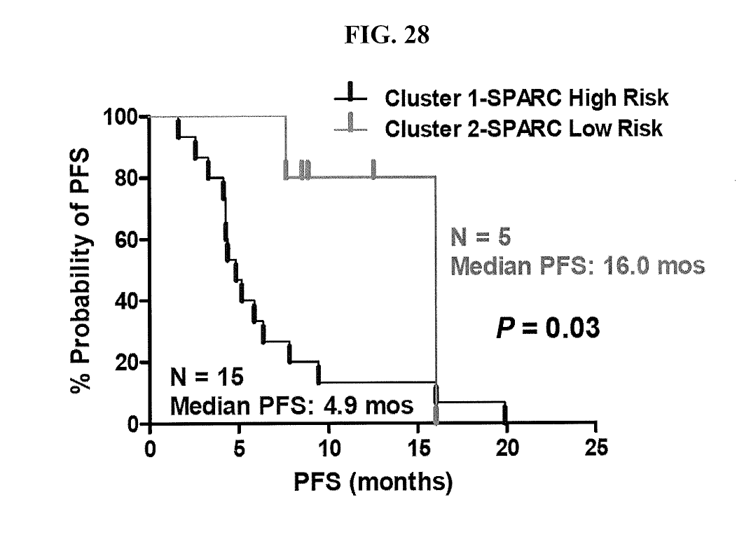

SPARC SMS in primary tumors did not correlate with clinical outcomes. However, the SPARC SMS of metastatic tumors correlated with outcomes such that the SMS data was sufficient to discriminate between a high risk cluster (Cluster 1 in FIG. 27) and a low risk cluster (Cluster 2 in FIG. 27). In this analysis, high and low risk clusters were defined according to PFS (median PFS was 16.0 months in the low risk cluster versus 4.9 months in the high risk cluster, p=0.03, log-rank). The predictive value of the SMS's associated with the two clusters is depicted graphically in FIG. 28. As can be seen, the SPARC SMS associated with cluster two correlates with a notably higher percentage of patients experiencing longer periods of PFS.

This example demonstrates that SPARC SMS may be used to identify high and low risk TNMBC patients.

### Example 12

This example describes the predictive value of comparing SPARC SMS's obtained from biopsies of primary tumors and biopsies of metastatic tumors in patients with TNMBC. The analysis was performed on 17 paired primary and metastatic biopsies which were taken from 15 of the first line patients discussed in Example 10 (multiple metastatic biopsies were taken from two of the patients).

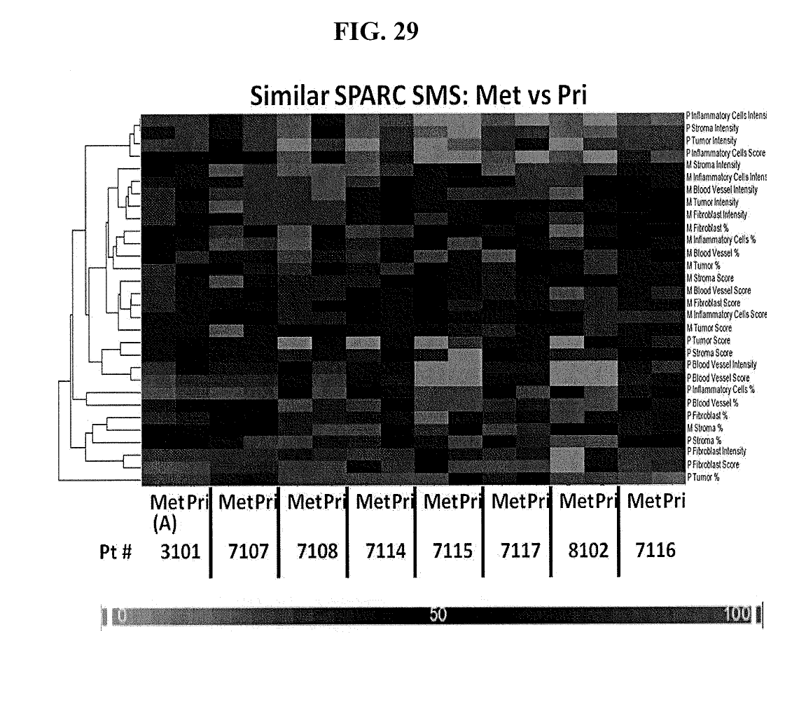

SMS's were obtained from paired paraffin-embedded biopsies from primary tumor and metastatic sites according to the IHC method previously described (See Example 6). The multivariable IHC data were then analyzed using GeneSpring® (Agilent Technologies, Santa Clara, Calif.) and Nexus® (Biodiscovery, Los Angeles Calif.) analysis programs. In 8 of the paired biopsies, the SMS for the metastatic biopsy was found to be similar to the SMS obtained for the primary biopsy. FIG. 28 presents a heat diagram for these 8 “similar” patients in which the general similarities between the SMS for the primary and metastatic biopsies are visible. In the remaining 9 pairs, the SMS of the metastatic biopsies and the primary biopsies were dissimilar. The general dissimilarities can be seen in the heat diagram for these 9 “dissimilar” patients presented in FIG. 29. Data from one of the patients (patient 3101) was excluded from subsequent analysis due to the fact that one of the patient's metastatic biopsies was similar to its primary counterpart, and the other metastatic biopsy was dissimilar.

ORR data was available for 14 of the patients. Of these 14, those with metastatic SMS's that were similar to their primary counterparts had better response to treatment with nab-paclitaxel/carboplatin/bevacizumab than those with metastatic SMS's dissimilar to their primary counterparts. In the “similar” group, 3 of 7 patients (43%) showed a complete response (CR), and the remaining 4 patients (57%) showed a partial response (PR). This can be contrasted with the results for the “dissimilar” group in which no patients showed a complete response (CR), 5 of 7 (71%) showed a partial response (PR) and in 2 of 7 patients the disease remained stable (SD). The responses for each patient are summarized in Table 26, below.

TABLE 26
Response rates for TNMBC patients to nab-
paclitaxel/carboplatin/bevacizumab treatment.

| | | |
|---|---|---|
| Patient Number | Best Response | PFS (Days) |

Similar SPARC SMS: Met vs Pri

| | | |
|---|---|---|
| 7107 | CR | 194 |
| 7108 | PR | 131 |
| 7114 | PR | 79 |
| 7115 | PR | 270 |
| 7116 | CR | 127 |
| 7117 | CR | 131 |
| 8102 | PR | 381 |

Dissimilar SPARC SMS: Met vs Pri

| | | |
|---|---|---|
| 6102 | PR | 148 |
| 6104 | SD | 179 |
| 6106 | SD | 260 |
| 7103 | PR | 233 |
| 7105 | PR | 51 |
| 7106 | PR | 487 |
| 7110 | PR | 101 |

Since patients with primary tumors exhibiting SPARC SMS's different from their metastatic tumors show reduced response to treatment, these data suggest that significant alterations in these tumor's pathological characteristics occurred following metastasis.

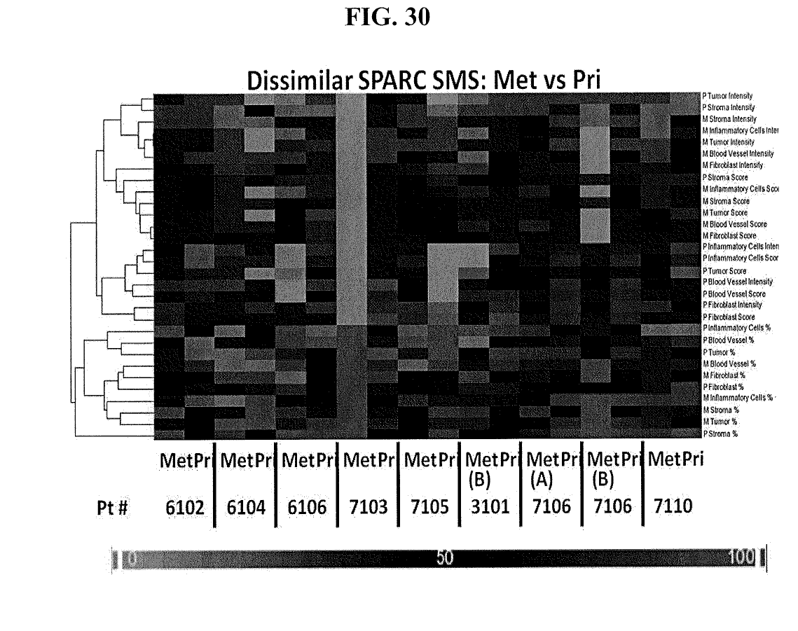

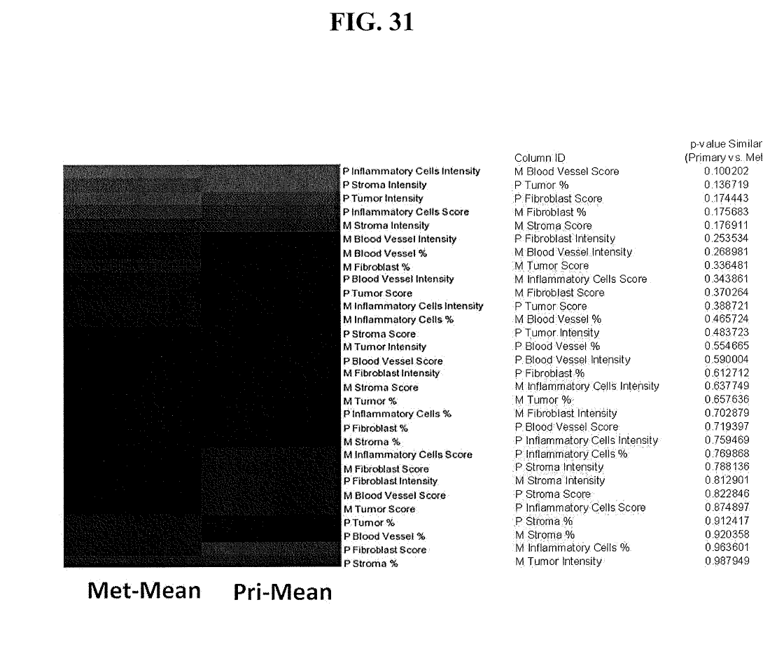

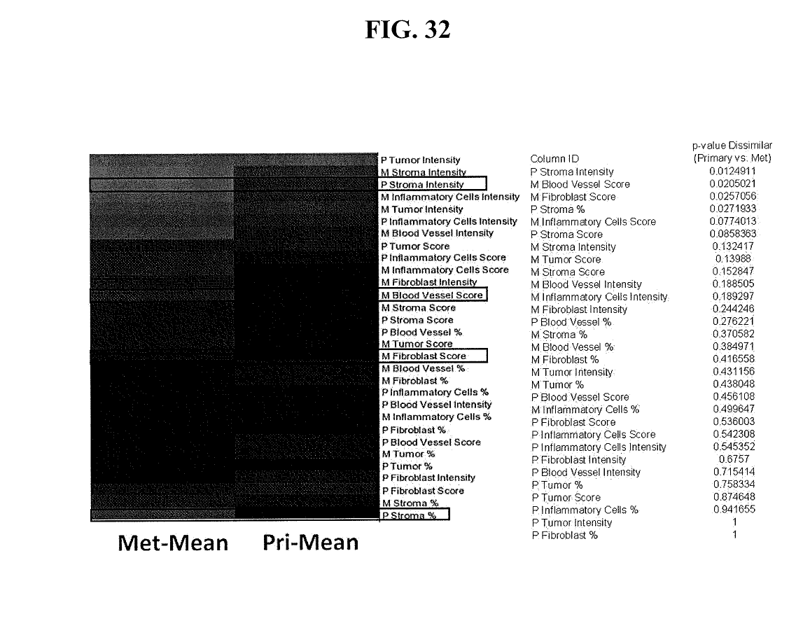

Next the SMS's for primary and metastatic tumors in were compared to determine which specific SMS components were different in primary and metastatic tumors. To facilitate comparison, a mean value for metastatic biopsies (Met-Mean) and primary biopsies (Pri-Mean) was determined for each group. For “similar” patients, the Met-Mean values were not significantly different from the Pri-Mean values for any of the SMS components (see FIG. 30). However, in the “dissimilar” group, significant differences between Met-Mean and Pri-Mean were observed in polyclonal antibody stained Stroma Intensity (P=0.012), monoclonal antibody stained Blood Vessel Score (P=0.021), monoclonal antibody stained Fibroblast Score (P=0.026), and for polyclonal antibody stained Stroma % (P=0.027) (see FIG. 31). Without being bound by any particular theory, the fact that changes in SPARC expression in metastatic tumors were apparent in the stroma, blood vessels and fibroblasts, but not in tumor cells, is consistent with the known presence of SPARC in the extracellular matrix and its role in angiogenesis.

The preceding example demonstrates that a comparison of SPARC SMS's between primary tumors and metastatic tumors can be a useful for predicting a patient's likely response to treatment with nab-paclitaxel/carboplatin/bevacizumab.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of treating a tumor in a mammal with a chemotherapeutic regimen comprising:
(a) determining a SPARC microenvironment signature (SMS) of the mammal, wherein the SMS is then compared to a predefined SMS;
(b) quantifying plasma SPARC in the mammal; and
(c) quantifying plasma lactate dehydrogenase (LDH) in the mammal; and
(d) administering a therapeutically effective amount of the chemotherapeutic regimen if two or more of the following conditions is met: SMS satisfies the criteria of a predetermined SMS, circulating SPARC is elevated as compared to a negative control, and plasma LDH is elevated as compared to a negative control.

2. A method of predicting a response to a chemotherapeutic regimen in a mammal comprising:
(a) determining a SPARC microenvironment signature (SMS) of the mammal, wherein the SMS is then compared to a predefined SMS;
(b) quantifying plasma SPARC in the mammal; and
(c) quantifying plasma lactate dehydrogenase (LDH) in the mammal,
wherein a positive response to the chemotherapeutic regimen is predicted if two or more of the following conditions are met: SMS satisfies the criteria of a low-risk SMS, circulating SPARC is not elevated as compared to a negative control, and plasma LDH is not elevated as compared to a negative control; and a negative response to the chemotherapeutic regimen is predicted if two or more of the following conditions are met: SMS satisfies the criteria of a high-risk SMS, circulating SPARC is elevated as compared to a negative control, and plasma LDH is elevated as compared to a negative control.

3. A method of determining a prognosis of a tumor in a mammal comprising:
(a) determining a SPARC microenvironment signature (SMS) of the mammal, wherein the SMS is then compared to a predefined SMS;
(b) quantifying plasma SPARC in the mammal; and
(c) quantifying plasma lactate dehydrogenase (LDH) in the mammal,
wherein a positive prognosis is determined if two or more of the following conditions are met: SMS satisfies the criteria of a low-risk SMS, circulating SPARC is not elevated as compared to a negative control, and plasma LDH is not elevated as compared to a negative control; and a negative prognosis is determined if two or more of the following conditions are met: SMS satisfies the criteria of a high-risk SMS, circulating SPARC is elevated as compared to a negative control, and plasma LDH is elevated as compared to a negative control.

4. The method of claim 1, wherein the tumor is breast cancer and the predefined SMS comprises immunostaining with a composite profile with at least 82% of the stroma staining positive with a first antibody and at least a Fibroblast Score of 87, Fibroblast Intensity of 68, Tumor Intensity of 49, Inflammatory Cells Intensity of 42, Inflammatory Cells Score of 67, Blood Vessel % of 68, Tumor Score of 76, Blood Vessel Intensity of 46, Fibroblast % of 51, Blood Vessel Intensity of 75, Inflammatory Cells % of 59, and Stroma Score of 62 staining with a second antibody, wherein the therapy is a regimen comprising nab-paclitaxel and the tumor is pancreatic cancer.

5. The method of claim 1, wherein the circulating SPARC is elevated as compared to a negative control if it is present at more than about 366 ng/mL.

6. The method claim 5, wherein the circulating SPARC is elevated as compared to a negative control if it is present at more than about 431 ng/mL.

7. The method claim 6, wherein the circulating SPARC is elevated as compared to a negative control if it is present at more than about 495 ng/mL.

8. The method of claim 1, wherein LDH is elevated as compared to a negative control if it is greater than about 212 IU/mL.

9. The method of claim 8, wherein LDH is elevated as compared to a negative control if it is greater than about 250 IU/mL.

10. The method of claim 9, wherein LDH is elevated as compared to a negative control if it is greater than about 287 IU/mL.

11. The method of claim 1, wherein plasma SPARC and LDH are elevated as compared to a negative control.

12. The method of claim 1, wherein plasma SPARC and LDH are not elevated as compared to a negative control.

13. The method of claim 1, wherein plasma SPARC is elevated as compared to a negative control, and wherein the SMS satisfies the criteria of a high-risk SMS.

14. The method of claim 1, wherein plasma SPARC is not elevated as compared to a negative control, and wherein the SMS satisfies the criteria of a low-risk SMS.

15. The method of claim 1, wherein plasma LDH is elevated as compared to a negative control, and wherein the SMS satisfies the criteria of a high-risk SMS.

16. The method of claim 1, wherein plasma LDH is not elevated as compared to a negative control, and wherein the SMS does not satisfy the criteria of a high-risk SMS.

17. The method of claim 1, wherein SMS satisfies the criteria of a high-risk SMS, circulating SPARC is elevated as compared to a negative control, and plasma LDH is elevated as compared to a negative control.

18. The method of claim 1, wherein SMS satisfies the criteria of a low-risk SMS, circulating SPARC is not elevated as compared to a negative control, and plasma LDH is not elevated as compared to a negative control.

19. The method of claim 1, wherein the tumor is selected from the group consisting of oral cavity tumors, pharyngeal tumors, digestive system tumors, respiratory system tumors, bone tumors, cartilaginous tumors, bone metastases, sarcomas, skin tumors, melanoma, breast tumors, genital system tumors, urinary tract tumors, orbital tumors, brain and central nervous system tumors, gliomas, endocrine system tumors, thyroid tumors, esophageal tumors, gastric tumors, small intestinal tumors, colonic tumors, rectal tumors, anal tumors, liver tumors, gall bladder tumors, pancreatic tumors, laryngeal tumors, tumors of the lung, bronchial tumors, non-small cell lung carcinoma, small cell lung carcinoma, uterine cervical tumors, uterine corpus tumors, ovarian tumors, vulvar tumors, vaginal tumors, prostate tumors, prostatic carcinoma, testicular tumors, tumors of the penis, urinary bladder tumors, tumors of the kidney, tumors of the renal pelvis, tumors of the ureter, head and neck tumors, parathyroid cancer, Hodgkin's disease, Non-Hodgkin's lymphoma, multiple myeloma, leukemia, acute lymphocytic leukemia, chronic lymphocytic leukemia, acute myeloid leukemia, and chronic myeloid leukemia.

20. The method of claim 19, wherein the tumor is a pancreatic cancer.

21. The method of claim 1, wherein the mammal is a human.

22. The method of claim 1, wherein the chemotherapeutic regimen comprises paclitaxel.

23. The method of claim 22, wherein the chemotherapeutic regimen comprises nab-Paclitaxel.

24. A method of treating a metastatic cancer in a mammal with a chemotherapeutic regimen comprising:
(a) determining a SPARC microenvironment signature (SMS) of the mammal's primary tumor,
(b) determining a SMS of a metastatic tumor from said primary tumor in the same mammal,
(b) determining if the SMS of the metastatic tumor is sufficiently similarity to the SMS of the primary tumor; and
(c) administering a therapeutically effective amount of the chemotherapeutic regimen if the if the SMS of the metastatic tumor sample is sufficiently similar to the SMS of the primary tumor sample.

25. A method of treating a tumor in a mammal with a chemotherapeutic regimen comprising:
(a) determining a SPARC microenvironment signature (SMS) of the mammal's primary tumor and a SMS of a metastatic tumor from said primary tumor in said mammal;
(b) quantifying plasma SPARC in the mammal; and
(c) quantifying plasma lactate dehydrogenase (LDH) in the mammal; and
(d) administering a therapeutically effective amount of the chemotherapeutic regimen if two or more of the following conditions is met: the SMS of a primary tumor is sufficiently similar to the SMS of the metastatic tumor; plasma SPARC is not elevated as compared to a negative control, and plasma LDH is not elevated as compared to a negative control.

26. A method of predicting a response to a chemotherapeutic regimen in a mammal comprising:
(a) determining a SPARC microenvironment signature (SMS) of the mammal's primary tumor and a SMS of a metastatic tumor from said primary tumor in said mammal;
(b) quantifying plasma SPARC in the mammal; and
(c) quantifying plasma lactate dehydrogenase (LDH) in the mammal,
wherein a positive response to the chemotherapeutic regimen is predicted if two or more of the following conditions are met: the SMS of a primary tumor is sufficiently similar to the SMS of the metastatic tumor; plasma SPARC is not elevated as compared to a negative control, plasma LDH is not elevated as compared to a negative control; and a negative response to the chemotherapeutic regimen is predicted if two or more of the following conditions are met: the SMS of a primary tumor is not sufficiently similar to the SMS of the metastatic tumor, plasma SPARC is elevated as compared to a negative control, and plasma LDH is elevated as compared to a negative control.

27. A method of determining a prognosis of a tumor in a mammal comprising:
(a) determining a SPARC microenvironment signature (SMS) of the mammal's primary tumor and a SMS of a metastatic tumor from said primary tumor in said mammal;
(b) quantifying plasma SPARC in the mammal; and
(c) quantifying plasma lactate dehydrogenase (LDH) in the mammal,
wherein a positive prognosis is determined if two or more of the following conditions are met: the SMS of a primary tumor is sufficiently similar to the SMS of the metastatic tumor, plasma SPARC is not elevated as compared to a negative control, and plasma LDH is not elevated as compared to a negative control; and a negative prognosis is determined if two or more of the following conditions are met: the SMS of a primary tumor is not sufficiently similar to the SMS of the metastatic tumor; plasma SPARC is elevated as compared to a negative control, and plasma LDH is elevated as compared to a negative control.

28. The method of claim 24, wherein the SMS of the metastatic tumor is sufficiently similar to the SMS of the primary tumor if there are no statistically significant differences between the value of any SMS component measured in the metastatic tumor and the value of the same SMS component measured in the primary tumor sample.

29. The method of claim 24, wherein the SMS of the metastatic tumor sample is not sufficiently similar to the SMS of the primary tumor if there are statistically significant differences between each of the following SMS components measured in the primary tumor and the metastatic tumor: stroma intensity and % positive stroma determined with the polyclonal antibody and blood vessel score and the fibroblast score determined with the monoclonal antibody.

30. The method of claim 24, wherein the SMS of the metastatic tumor is sufficiently similar to the SMS of the primary tumor if there is a statistically significant correlation between the SMS component values of the metastatic tumor sample with the corresponding SMS component values from the primary tumor sample.

31. The method of claim 25, wherein the circulating SPARC is elevated as compared to a negative control if it is present at more than about 366 ng/mL.

32. The method of claim 25, wherein the circulating SPARC is elevated as compared to a negative control if it is present at more than about 431 ng/mL.

33. The method of claim 25, wherein the circulating SPARC is elevated as compared to a negative control if it is present at more than about 495 ng/mL.

34. The method of claim 25, wherein LDH is elevated as compared to a negative control if it is greater than about 212 IU/mL.

35. The method of claim 25, wherein LDH is elevated as compared to a negative control if it is greater than about 250 IU/mL.

36. The method of claim 25, wherein LDH is elevated as compared to a negative control if it is greater than about 287 IU/mL.

37. The method of claim 25, wherein plasma SPARC and LDH are elevated as compared to a negative control.

38. The method of claim 25, wherein plasma SPARC and LDH are not elevated as compared to a negative control.

39. The method of claim 24, wherein the chemotherapeutic regimen comprises a nanoparticulate albumin bound paclitaxel.

40. The method of claim 24, wherein the tumor is selected from the group consisting of oral cavity tumors, pharyngeal tumors, digestive system tumors, respiratory system tumors, bone tumors, cartilaginous tumors, bone metastases, sarcomas, skin tumors, melanoma, breast tumors, genital system tumors, urinary tract tumors, orbital tumors, brain and central nervous system tumors, gliomas, endocrine system tumors, thyroid tumors, esophageal tumors, gastric tumors, small intestinal tumors, colonic tumors, rectal tumors, anal tumors, liver tumors, gall bladder tumors, pancreatic tumors, laryngeal tumors, tumors of the lung, bronchial tumors, non-small cell lung carcinoma, small cell lung carcinoma, uterine cervical tumors, uterine corpus tumors, ovarian tumors, vulvar tumors, vaginal tumors, prostate tumors, prostatic carcinoma, testicular tumors, tumors of the penis, urinary bladder tumors, tumors of the kidney, tumors of the renal pelvis, tumors of the ureter, head and neck tumors, parathyroid cancer, Hodgkin's disease, Non-Hodgkin's lymphoma, multiple myeloma, leukemia, acute lymphocytic leukemia, chronic lymphocytic leukemia, acute myeloid leukemia, and chronic myeloid leukemia.
